(19) United States
(12) Patent Application Publication
(10) Pub. No.: US 2022/0133761 A1
(43) Pub. Date: May 5, 2022

(54) NEUTROPHIL APOPTOSIS INDUCED BY NANOPARTICLES FOR TREATING INFLAMMATORY DISEASES

(71) Applicant: WASHINGTON STATE UNIVERSITY, Pullman, WA (US)

(72) Inventors: Zhenjia WANG, Spokane, WA (US); Canyang ZHANG, Pullman, WA (US)

(21) Appl. No.: 17/518,031

(22) Filed: Nov. 3, 2021

Related U.S. Application Data
(60) Provisional application No. 63/109,773, filed on Nov. 4, 2020.

(51) Int. Cl. A61K 31/704; A61K 47/64; A61K 47/69; A61P 29/00

(52) U.S. Cl. CPC A61K 31/704; A61P 29/00; A61K 47/6929; A61K 47/643

(57) ABSTRACT

Disclosed are methods and compounds to selectively target the apoptosis pathway in proinflammatory neutrophils using Topoisomerase nanoparticles (NPs). The design of the disclosed nanoparticles (NPs) allows controlled release of DOX inside neutrophils, thus avoiding systemic toxicity. One such beneficial method for treating a subject neutrophil-inflammatory infection or tissue injury response includes administering a topoisomerase-conjugated albumin protein nanoparticles (NPs) composition to the subject, wherein the composition includes an effective amount so as to induce apoptosis of neutrophils, wherein the neutrophil induced inflammatory response is lower following the administration of the composition.

### CROSS-REFERENCE TO RELATED APPLICATIONS

The instant application claims under 35 U.S.C. § 119, the priority benefit of U.S. Provisional Application No. 63/109,773, entitled, “NEUTROPHIL APOPTOSIS INDUCED BY NANOPARTICLES IMPROVED THE THERAPIES OF INFLAMMATORY DISEASES,” filed Nov. 4, 2020, of which is incorporated herein by reference in its entirety.

### GOVERNMENT INTERESTS

This invention was made with government support under Grant/Contract Number R01 GM116823, awarded by the National Institutes of Health. The government has certain rights in the invention.

### FIELD OF THE INVENTION

The invention relates to methods for inducing neutrophil apoptosis for treating activated neutrophil inflammatory diseases.

### BACKGROUND OF THE INVENTION

Polymorphonuclear neutrophils (PMNs) are the most abundant white blood cells (50-70%) in humans, playing a central role in the innate immune response to infections or tissue injury. It is known in the field that their defense mechanism involved in neutrophil infiltration and pro-inflammatory responses may be potentially detrimental to the host if neutrophils are dysregulated. Exaggerated activation and uncontrolled infiltration of neutrophils cause inflammatory and autoimmune diseases, such as acute lung inflammation/injury (ALI), ischemia/reperfusion, rheumatoid arthritis, and sepsis. Anti-inflammatory agents are usually used to treat these diseases, for example, nonsteroidal anti-inflammatory drugs and anti-cytokine therapies. The off-targeting of these therapies may cause the systemic immune suppression leading to severe side effects and susceptibility to infection, Depletion of neutrophils in blood and the bone marrow by administration of antibodies shows reduced inflammatory responses, indicating that targeting neutrophils is an applicable strategy to alleviate inflammatory disorders. However, total loss of the immune sentinel neutrophils using antibodies renders the vulnerability to infections and impairs innate and adaptive immune systems. Therefore, it is needed to develop new strategies to specifically target inflammatory neutrophils.

Neutrophils have a short lifespan in circulation (8-20 h), and their lifespan is precisely regulated by apoptosis. Apoptosis is a process of programmed cell death to maintain constant neutrophil numbers in circulation. Neutrophils undergo constitutive or spontaneous apoptosis that is a mechanism to preserve the immune homeostasis Inflammation caused by harmful stimuli (microorganisms or damaged tissues) rapidly increases the numbers of neutrophils in blood and their longevity extends. Subsequently, neutrophils are activated for transmigration and promote the cytokine release. Delayed/impaired apoptosis of neutrophils initiates acute and chronic inflammatory disorders, such as acute lung inflammation/injury, sepsis and ischemic stroke. Therefore, specifically targeting inflammatory neutrophils to promote their apoptosis in time may be a strategy for improved therapies of inflammatory diseases. Doxorubicin (DOX) is a widely-used drug in cancer therapy that is known to show severe cardiac toxicity and resultant increases in inflammation, such as when utilized for cancer treatment. In addition, intercalation of DOX into DNA double helices inhibits the progression of topoisomerase II, causing DNA damage to induce cell death.

Background information on a method for treating neutrophil-mediated inflammatory diseases but that does not teach nanoparticles conjugated to, for example, Doxorubicin, at particular novel dose ranges for such treatment, is described and claimed in U.S. Pat. No. 9,872,839 entitled “COMPOSITIONS AND METHODS FOR DIAGNOSING OR TREATING NEUTROPHIL-MEDIATED INFLAMMATORY DISEASE,” issued Jan. 23, 2018, to Wang et al., including the following, “Disclosed are nanoparticle compositions comprising nano particles prepared from denatured, cross-linked albumin and a therapeutic agent for treating a neutrophil-mediated inflammation, and methods of treating neutrophil-mediated inflammation using the compositions.”

Accordingly, there is a need to precisely control neutrophil apoptosis to resolve inflammation and return immune homeostasis. The methods herein address such a need by way of in situ novel low doses of doxorubicin (DOX) conjugated to albumin protein nanoparticles (NPs) so as to selectively target inflammatory neutrophils for intracellular delivery of DOX that induces neutrophil apoptosis.

### SUMMARY OF THE INVENTION

The methods disclosed herein beneficially utilize a composition of doxorubicin (DOX)-conjugated human or bovine albumin protein nanoparticles (NPs). The DOX release itself is triggered by acidic environments in neutrophils, subsequently inhibiting neutrophil transmigration and inflammatory responses. In particular, the composition is administered in-situ so as to selectively target (i.e., bind to and be internalized) inflammatory neutrophils for intracellular delivery of DOX that induces neutrophil apoptosis.

The disclosure having supporting enabling data, provides as an aspect, a method of treating a subject with doxorubicin (DOX) to prevent an activated neutrophil-inflammatory response and transmigration, including: administering to the subject, a composition comprising doxorubicin (DOX)-conjugated albumin protein nanoparticles (NPs), wherein the composition includes an effective amount of 0.1 mg/kg up to 10 mg/kg of the doxorubicin (DOX)-conjugated albumin protein nanoparticles (NPs) in vivo, wherein the protein nanoparticles (NPs) selectively bind to and are internalized by the activated neutrophils for intracellular delivery of the doxorubicin (DOX) so as to induce apoptosis of the activated neutrophils; and wherein a neutrophil induced inflammatory response and transmigration is lower following the administration of the 0.1 mg/kg up to 10 mg/kg of the doxorubicin (DOX)-conjugated albumin protein nanoparticles (NPs).

### BRIEF DESCRIPTION OF DRAWINGS

FIG. 1A graphically shows nanoparticle targeting of proinflammatory neutrophils to induce their apoptosis for treatment of inflammatory diseases. In particular, FIG. 1A shows a therapeutic process, as disclosed herein, of DOX-conjugated BSA NPs includes neutrophil uptake of NPs in situ and cell apoptosis to prevent neutrophil transmigration and inflammatory responses.

FIG. 1B graphically shows nanoparticle targeting of proinflammatory neutrophils to induce their apoptosis for treatment of inflammatory diseases. In particular, FIG. 1B shows DOX conjugated to BSA via a hydrazone bond, followed by aggregating BSA conjugates to form DOX-hyd-BSA NPs, and DOX release from NPs triggered by low pH in neutrophils to promote neutrophil apoptosis.

FIG. 2A shows synthetic routes of DOX-hyd-BSA, as disclosed herein.

FIG. 2B shows H NMR spectra of HOOC-PEG-hyd in d-CDCl₃ and of HOOC-PEG-hyd-DOX in D₂O.

FIG. 3A shows particle size of BSA NPs, DOX-ab-BSA NPs and DOX-hyd-BSA NPs measured by dynamic light scattering in PBS at a pH of 7.4.

FIG. 3B shows additional characterization of DOX-conjugated BSA NPs and DOX release from NPs at acidic condition to cause cell death. In particular, FIG. 3B shows TEM images of DOX-hyd-BSA NPs. Scale bar: 500 nm for larger image and 100 nm for inset.

FIG. 3C shows tunneling microscopy (TEM) images of BSA NPs.

FIG. 3D shows tunneling microscopy (TEM) images of DOX-ab-BSA NPs.

FIG. 3E shows the Zeta-potential of BSA NPs, DOX-ab-BSA NPs and DOX-hyd-BSA NPs in PBS at different pH values.

FIG. 3F shows stability of BSA NPs, DOX-ab-BSA NPs and DOX-hyd-BSA NPs in PBS with 20% FBS at pH 7.4.

FIG. 4A shows characterization of DOX-conjugated BSA NPs and DOX release from NPs at acidic condition to cause cell death. In particular, FIG. 4A shows UV-Vis spectra of their supernatants after NPs were incubated in PBS at different pH for 2 h. It shows that DOX-hyd-BSA NPs can release DOX from BSA NPs at acidic environments.

FIG. 4B shows additional characterization of DOX-conjugated BSA NPs and DOX release from NPs at acidic condition to cause cell death. In particular, FIG. 4B shows Cumulative DOX release from DOX-ab-BSA NPs or DOX-hyd-BSA NPs in PBS at pH 7.4, 6.5 or 5.0. It shows that DOX-BSA NPs completely released DOX in 30 h at pH 5.

FIG. 4C shows additional characterization of DOX-conjugated BSA NPs and DOX release from NPs at acidic conditions. In particular, FIG. 4B shows cell death (viability) of differentiated HL-60 cells (neutrophil-like cells) induced by DOX. Cells were treated for 24 h at different concentrations of DOX. Data are shown as mean±s.d. (n=6 independent experiments).

FIG. 4D shows cytotoxicity of BSA NPs to HL60 cells. The cells were incubated with BSA NPs for 24 h at different concentrations of NPs. Data are shown as mean±s.d. (n=6 independent experiments). The result shows that BSA NPs were not toxic.

FIG. 5A shows intravital microscopy images of mouse cremaster muscle venules, wherein such images indicate that neutrophil activation was associated with upregulation of Fey receptors.

FIG. 5B shows the percentage of co-staining between anti-mouse CD16/32 and anti-mouse LY-6G based on intravital images of FIG. 5A.

FIG. 5C shows resultant uptake data of DOX-hyd-BSA NPs by differentiated HL60 cells after they (at 10⁶ cells/mL) were incubated with DOX-hyd-BSA NPs at different concentrations of DOX for 2 h.

FIG. 5D shows time course of DOX uptake by differentiated HL60 cells after they (at 10⁶ cells/mL) were incubated with DOX-hyd-BSA NPs (at 30 m/mL of DOX).

FIG. 5E shows confocal laser scanning microscopy (CLSM) images of blood neutrophils from healthy mice or LPS-challenged mice.

FIG. 5F shows uptake of BSA NPs by blood leukocytes analyzed by flow cytometry.

FIG. 6A shows a confocal fluorescence microscopy image of cells treated with free DOX or DOX-hyd-BSA NPs contained staining of Annexin-V-FITC, and less staining of 7AAD to indicate that cells were alive.

FIG. 6B shows apoptosis flow cytometry analysis, wherein the analysis revealed that free DOX and DOX-hyd-BSA NPs caused cell apoptosis at 73% and 89%, respectively, whereas there were only 20% apoptotic cells after treatment with DOX-ab-BSA NPs.

FIG. 6C also shows the analysis like FIG. 6B, wherein the analysis revealed that free DOX and DOX-hyd-BSA NPs caused cell apoptosis at 73% and 89%, respectively, whereas there were only 20% apoptotic cells after treatment with DOX-ab-BSA NPs.

FIG. 7A shows a confocal fluorescence microscopy image that indicates that nearly 100% cells showed co-staining of DOX and TUNEL after the cells were treated with free DOX.

FIG. 7B shows quantification data of co-staining of DOX and TUNEL in cells, wherein such data indicates that DOX-hyd-BSA NPs effectively induces neutrophil apoptosis.

FIG. 7C shows a graphical representation of the experimental protocol related to the data of FIG. 8A and FIG. 8B in an acute lung inflammation mouse model. Intratracheal administration of LPS causes local lung acute inflammation and subsequently neutrophils transmigrate from blood to airspace in the lung. 24 h after i.v. injection of several DOX formulations, BALF was collected to assess neutrophil number and cytokines.

FIG. 8A shows neutrophil flow cytometry data, wherein upon treatment with DOX-hyd-BSA NPs at a very low dose of 0.2 mg/kg (DOX), neutrophils dramatically decreased compared to controls (e.g., free DOX- and DOX-ab-BSA NPs-treated groups).

FIG. 8B also shows neutrophil flow cytometry data, wherein upon treatment with DOX-hyd-BSA NPs at a very low dose of 0.2 mg/kg (DOX), neutrophils dramatically decreased compared to controls (e.g., free DOX- and DOX-ab-BSA NPs-treated groups).

FIG. 9A shows inflammatory factors, (TNF-α) decreased after treatment with DOX-hyd-BSA NPs compared to other treatments (such as, free DOX and DOX-ab-BSA NPs).

FIG. 9B shows inflammatory factors, (IL-1β) decreased after treatment with DOX-hyd-BSA NPs compared to other treatments (such as, free DOX and DOX-ab-BSA NPs).

FIG. 9C shows inflammatory factors, (IL-6) decreased after treatment with DOX-hyd-BSA NPs compared to other treatments (such as, free DOX and DOX-ab-BSA NPs).

FIG. 10A shows mouse survival rates in sepsis after treatments of NPs. 4 h after i.p. LPS (50 mg/kg) challenge to mice, mice were treated with PBS, free DOX and prodrug DOX-hyd-BSA NPs at 0.2 mg/kg of DOX, respectively.

FIG. 10B shows data of mouse body weights measured after treatments of PBS.

FIG. 10C shows data of mouse body weights measured after treatments of free DOX measurements.

FIG. 10D shows data of mouse body weights measured after treatments of DOX-hyd-BSA NPs.

FIG. 10E shows data of number of neutrophils in blood.

FIG. 10F shows concentrations of TNF-α's in blood.

FIG. 10G shows concentrations of IL-1β's in blood.

FIG. 10H shows concentrations of IL-6's in blood.

FIG. 10I shows data of number of neutrophils in BALF at 16 h and 72 h post LPS challenge.

FIG. 10J shows concentrations of TNF-α in BALF at 16 h and 72 h post LPS challenge.

FIG. 10K shows concentrations of IL-1β in BALF at 16 h and 72 h post LPS challenge.

FIG. 10L shows concentrations of IL-6 in BALF at 16 h and 72 h post LPS challenge.

FIG. 10M show a diagram of the experimental protocol to address whether DOX-conjugated BSA NPs impair neutrophil immune sentinel to the secondary infection. The mice were challenged with LPS (i.p., 50 mg/kg) or PBS (control). 4 h later, the LPS-challenged mice were i.v. treated with DOX-hyd-BSA NPs at 0.2 mg/kg of DOX. The control mice were not treated with LPS and NPs. 72 h later, all survival and control (healthy) mice were challenged with LPS (i.t., 10 mg/kg).

FIG. 10N shows data that t 84 h, BALF was collected to assess neutrophil number.

FIG. 10O shows data that t 84 h, BALF was collected to assess TNF-α concentrations.

FIG. 10P shows data that t 84 h, BALF was collected to assess IL-1β concentrations.

FIG. 10Q shows data that t 84 h, BALF was collected to assess IL-1β concentrations.

FIG. 11A shows MPO activity 4 h after (i.p.) LPS-challenge (50 mg/kg), (after mice were treated with PBS or DOX-hyd-BSA NPs).

FIG. 11B shows TNF-α activity 4 h after (i.p.) LPS-challenge (50 mg/kg), (after mice were treated with PBS or DOX-hyd-BSA NPs).

FIG. 11C shows IL-6 activity 4 h after (i.p.) LPS-challenge (50 mg/kg), (after mice were treated with PBS or DOX-hyd-BSA NPs).

FIG. 11D shows IL-1β activity 4 h after (i.p.) LPS-challenge (50 mg/kg), (after mice were treated with PBS or DOX-hyd-BSA NPs).

FIG. 12A shows mouse body weights of 3 Mice as the control group (healthy mice) as monitored following the graphical experiment protocol shown in FIG. 10M.

FIG. 12B shows mouse body weights of 7 mice (right) of the DOX-hyd-BSA NPs-treatment group (using a 0.2 mg (DOX)/kg dose) as monitored following the graphical experiment protocol shown in FIG. 10M.

FIG. 13 shows Toxicity of DOX-conjugated BSA NPs evaluated by histological analysis.

FIG. 14A shows the experimental design to examine the benefit of DOX-hyd-BSA NPs in cerebral I/R.

FIG. 14B shows that DOX-hyd-BSA NPs mitigated neutrophil-induced neuroinflammation in cerebral I/R mouse model so as to restore neurological functions. Specifically, FIG. 14B shows MPO activity in brain damaged tissues at 22 h post administration of PBS, free DOX, and DOX-hyd-BSA NPs.

FIG. 14C shows that DOX-hyd-BSA NPs mitigated neutrophil-induced neuroinflammation in cerebral I/R mouse model so as to restore neurological functions. Specifically, FIG. 14C shows TNF-α activity in brain damaged tissues at 22 h post administration of PBS, free DOX, and DOX-hyd-BSA NPs.

FIG. 14D shows DOX-hyd-BSA NPs mitigation of neutrophil-induced neuroinflammation in cerebral I/R mouse model so as to restore neurological functions. Specifically shows IL-1β activity in brain damaged tissues at 22 h post administration of PBS, free DOX, and DOX-hyd-BSA NPs.

FIG. 14E shows DOX-hyd-BSA NPs mitigation of neutrophil-induced neuroinflammation in cerebral I/R mouse model so as to restore neurological functions. Specifically, FIG. 14E shows IL-6 activity in brain damaged tissues at 22 h post administration of PBS, free DOX, and DOX-hyd-BSA NPs.

FIG. 14F shows Mouse neurological behavior scores after treatments with PBS, free DOX, and DOX-hyd-BSA NPs at 0.2 mg/kg of DOX.

FIG. 15 show novel data of uptake of DOX-hdy-BSA NPs by human neutrophils. Human neutrophils were incubated with DOX-hyd-BSA NPs (the concentration at 30 ug/ml) for 2 h, and then the cells were washed, and they were analyzed by flowcytometry.

FIG. 16A shows the molecular structure of DOX and a linker between DOX and human serum albumin.

FIG. 16B shows the synthesis of human serum albumin (HAS) conjugated with DOX. Specifically, FIG. 16B shows the detailed synthesis of DOX-hyd-HAS.

### DETAILED DESCRIPTION

In the description of the invention herein, it is understood that a word appearing in the singular encompasses its plural counterpart, and a word appearing in the plural encompasses its singular counterpart, unless implicitly or explicitly understood or stated otherwise. Furthermore, it is understood that for any given component or embodiment described herein, any of the possible candidates or alternatives listed for that component may generally be used individually or in combination with one another, unless implicitly or explicitly understood or stated otherwise. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

It is also to be understood that the nanoparticle compositions utilized herein may include a pharmaceutically acceptable excipient, vehicle, or carrier with which a compound as disclosed herein is administered.

“Treating” or “treatment” as used herein includes inhibiting a disease or disorder, i.e., arresting its development, relieving a disease or disorder, i.e., causing regression of the disorder; slowing progression of the disorder, and/or inhibiting, relieving, or slowing progression of one or more symptoms of the disease or disorder in a subject. Subjects treated may include human and non-human individuals, including warm blooded animals such as mammals afflicted with, or having the potential to be afflicted with one or more neutrophil-mediated diseases or disorders, including neutrophil-mediated inflammatory diseases.

It is also to be appreciated that the disclosure provides a pharmaceutical composition comprising the nanoparticle of the disclosure together with one or more pharmaceutically acceptable excipients, carriers, or vehicles, and optionally other therapeutic and/or prophylactic components.

A disorder, as disclosed herein, is characterized by functional impairment and a disruption to the body's normal function and structure. A disease is a pathological process that a caregiver is able to see, touch, and measure. An infection is often a first step to a disease, such as when undesirable microbes (e.g., bacteria or viruses) enter a body and begin to multiply.

An effective amount of a nanoparticle composition is an amount effective to provide the desired biological result of apoptosis of neutrophils. The result can be reduction and/or alleviation of inflammation that was produced from an infection or disease or disorder.

While the invention has been described in terms of its example embodiments, those skilled in the art will recognize that the invention can be practiced with modification within the spirit and scope of the appended claims. Accordingly, the present invention should not be limited to the embodiments as described above but should further include all modifications and equivalents thereof with the spirit and scope of the description provided herein.

### General Description

As generally understood by those of ordinary skill in the art, human neutrophils are the most abundant circulating leukocytes and contribute to acute and chronic inflammatory disorders. Neutrophil apoptosis is programed cell death to maintain immune homeostasis, but inflammatory responses to infections or tissue injury disrupt neutrophil death program leading to many diseases. Precise control of neutrophil apoptosis, as disclosed herein, is utilized to resolve inflammation to return immune homeostasis.

The methods disclosed herein beneficially utilize a composition of doxorubicin (DOX)-conjugated human or bovine albumin protein nanoparticles (NPs). It is to be emphasized that the composition of the present invention is not an anti-inflammatory drug. The DOX release itself is triggered by acidic environments (pH of between 4.0-6.5) in neutrophils, subsequently inhibiting neutrophil transmigration and inflammatory responses. In particular, the composition is administered in-situ so as to selectively target (i.e., bind to and be internalized) inflammatory neutrophils for intracellular delivery of DOX that induces neutrophil apoptosis.

In three disease models, (acute lung injury, sepsis, and stroke), DOX-conjugated NPs surprisingly and unexpectedly, significantly increased mouse survival in sepsis and prevented brain damage in cerebral ischemia/reperfusion, but the NPs did not suppress systemic immunity. Thereafter, the uptake of bovine albumin nanoparticles by human neutrophils in vitro was utilized to confirm that the nanoparticles can be efficiently taken up. In addition, synthesized human albumin nanoparticles were conjugated with DOX and the DOX release so as to show the release in the acidic environments.

### Specific Description

### Results

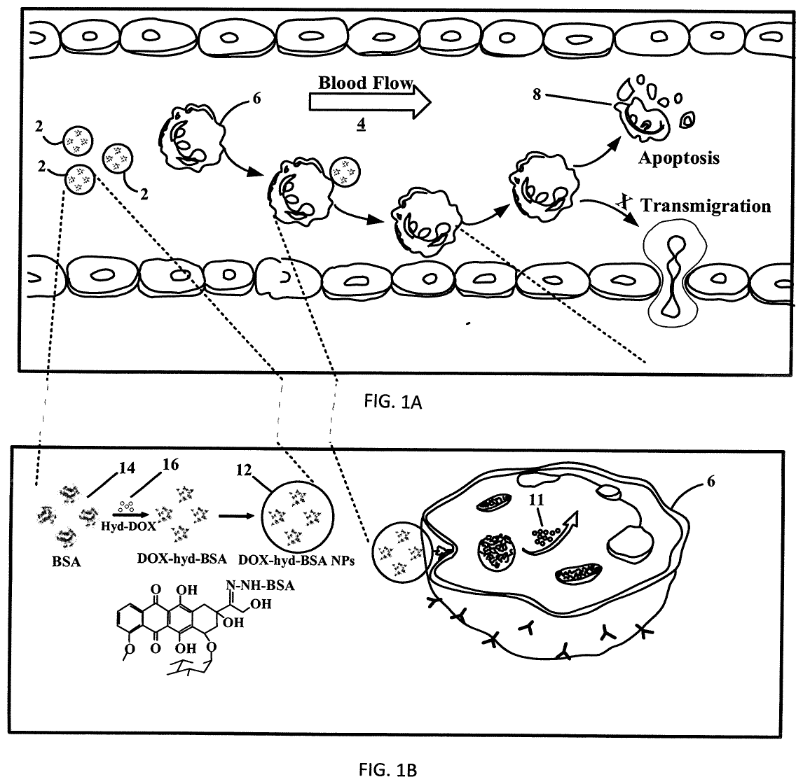

FIG. 1A shows a therapeutic process of DOX-conjugated BSA NPs includes neutrophil uptake of NPs in situ and cell apoptosis to prevent neutrophil transmigration and inflammatory responses and FIG. 1B shows DOX conjugated to BSA via a hydrazone bond, followed by aggregating BSA conjugates to form DOX-hyd-BSA NPs, and DOX release from NPs triggered by low pH in neutrophils to promote neutrophil apoptosis.

Specifically, in speaking to the drawings, FIG. 1A graphically illustrates an exemplary method of the nanoparticles 2 disclosed herein within a subject blood flow 4 of the present invention. Such nanoparticles 2 are thus designed to specifically target activated neutrophils 6 in vivo to deliver DOX to induce neutrophil apoptosis 8 so as to improve therapies of inflammatory disorders, as discussed infra.

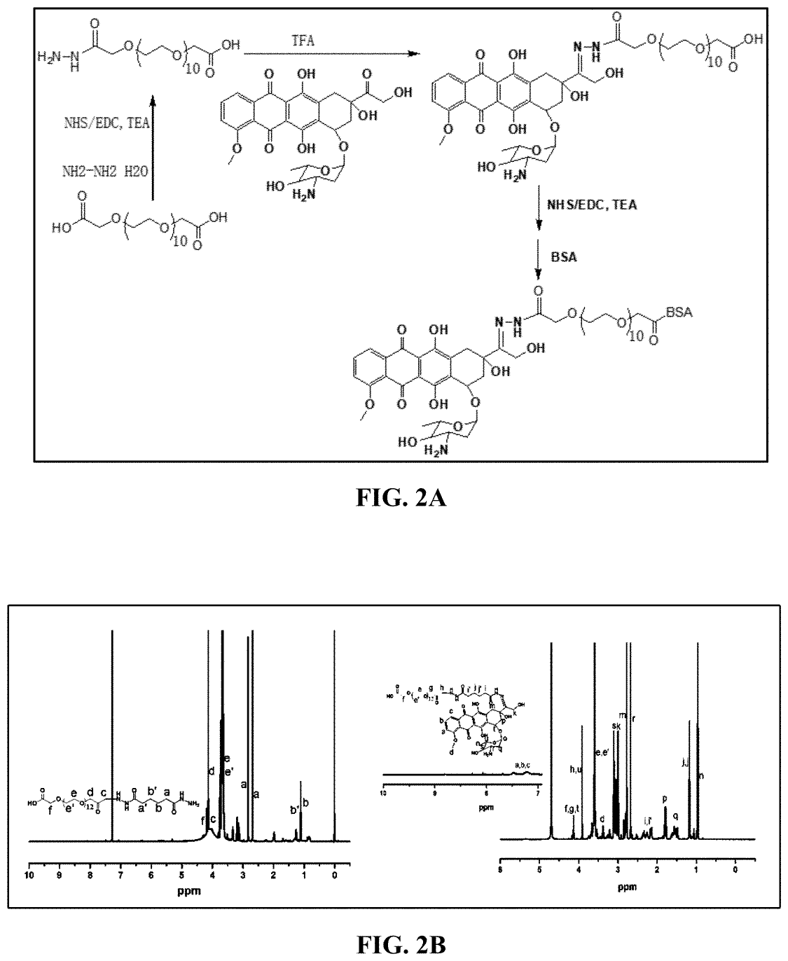

Moreover, FIG. 1B illustrates the delivery of DOX 11 in activated neutrophils 6, as shown in FIG. 1A, wherein a pH-sensitive (pH of between 4.0 to 7.4, more often between 4.0-6.5) DOX prodrug 12 was synthesized by conjugating DOX to, for example, bovine serum albumin (BSA) 14 via a hydrazone bond 16 (DOX-hyd-BSA). FIG. 2A shows the synthetic route of DOX-hyd-BSA and FIG. 2B shows NMR spectral characterization of chemical structures of DOX/BSA conjugates. Specifically, FIG. 2B shows H NMR spectra of HOOC-PEG-hyd in d-CDCl₃ (left) and of HOOC-PEG-hyd-DOX in D₂O (right).

To provide for the compositions disclosed herein, DOX was first conjugated to polyethylene glycol (PEG) through hydrazine to produce DOX-hyd-PEG, followed by conjugating to BSA. Beneficially, DOX itself does not migrate to the heart of the subject when conjugated to BSA, the result of which negates the deleterious effects to the heart when using such a drug. The BSA complexes were formed to nanoparticles (NPs) by desolvation, subsequently adding glutaraldehyde to crosslink BSA protein to make stable NPs. After intravenous administration of DOX-hyd-BSA NPs, the NPs specifically targeted activated neutrophils in circulation and were internalized when neutrophils responded to infections or tissue injury. Hydrazone bonds were cleaved by acid in neutrophil environments to release DOX molecules which were able to induce neutrophil apoptosis, thus mitigating neutrophil transmigration. In control, DOX was linked to BSA via a pH-insensitive amide bond (called DOX-ab-BSA), thus DOX was not released from BSA.

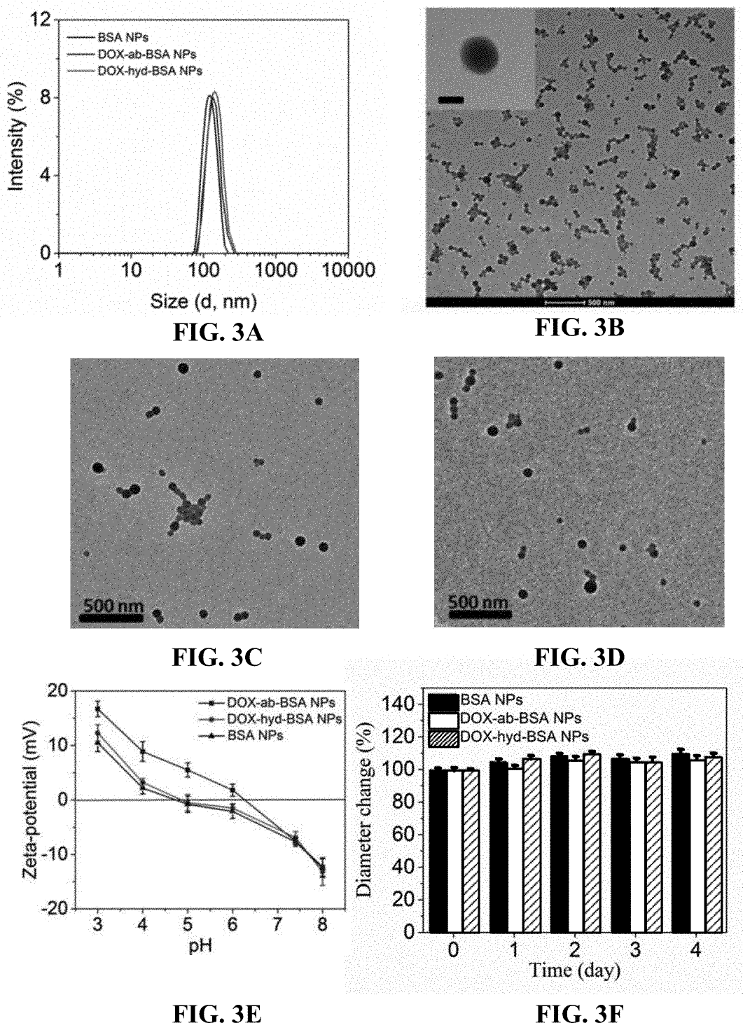

FIG. 3A-G show the characterization of DOX-conjugated BSA NPs and DOX release from NPs at acidic condition to cause cell death. The BSA NPs were generated by adding ethanol to induce the self-assembling of BSA into a nanoparticle, and subsequently BSA molecules were coupled using glutaraldehyde to form stable nanoparticle.

FIG. 3A, in particular, shows the characterizing of BSA NPs and two types of DOX-conjugated BSA NPs using dynamic light scattering (DLS), wherein resultant sizes were about 130 nm in diameter. FIG. 3B is a Transmission electron microscopy (TEM) image that shows a spherical shape of the nanoparticles (DOX-hyd-BSA) and FIG. 3C and FIG. 3D show BSA NPs and DOX-ab-BSA images respectively, wherein the size was consistent with the measurements by the DLS information shown in FIG. 3A.

The data shown in FIG. 3E shows the similarity of the surface charges of three types of BSA NPs, i.e., the Zeta-potential of BSA NPs, DOX-ab-BSA NPs and DOX-hyd-BSA NPs in PBS at different pH. Under alkaline conditions, the zeta-potential of NPs was negative due to the deprotonation of amine residues in NPs. Under acidic conditions, surface charges of NPs were switched to positive because amine residues in NPs were protonated. The trend of zeta-potentials of BSA NPs and DOX-hyd-BSA NPs was similar in acidic conditions, but DOX-ab-BSA NPs had the slightly higher surface charges because amine residues of DOX may contribute the additional protonation of amine residues. The results show that DOX may be released from DOX-hyd-BSA NPs at low pH. The serum stability of BSA-based NPs was evaluated in phosphate buffer solution (PBS) containing 20% FBS at pH 7.4. FIG. 3F shows the stability of BSA NPs, DOX-ab-BSA NPs and DOX-hyd-BSA NPs in PBS with 20% FBS at pH 7.4, wherein no aggregation (diameter change) was observed even after incubation for 4 days, thus displaying BSA NPs were very stable.

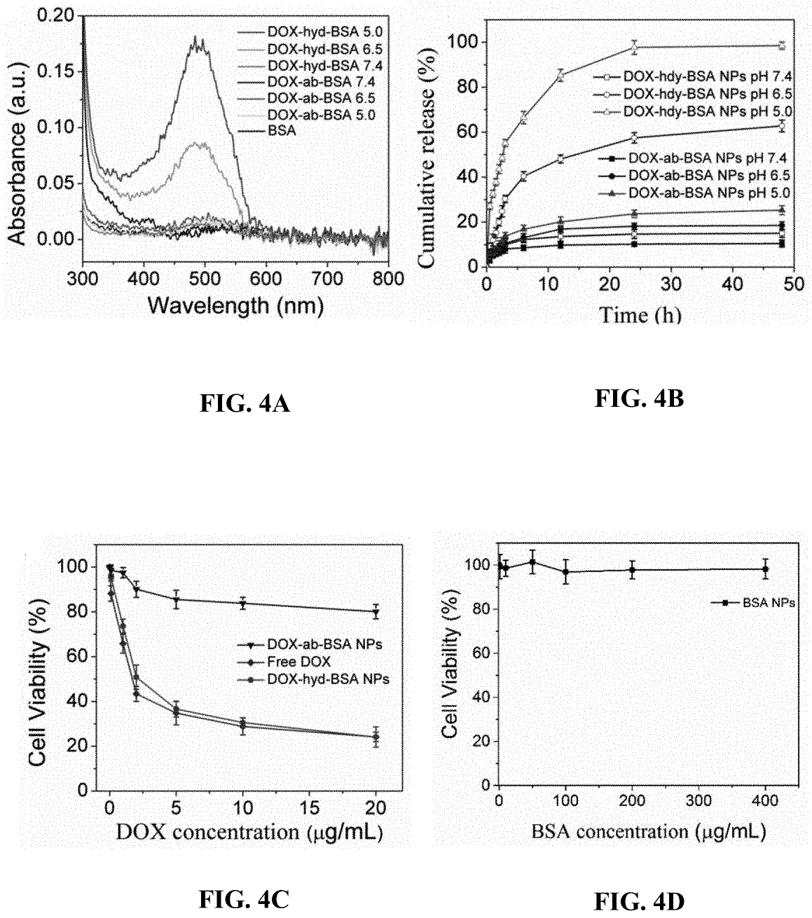

Next, BSA NPs were thereafter addressed to determine whether such NPs were responsive to acidic environments for DOX release. DOX-conjugated BSA NPs were incubated in PBS at pH 7.4 or at pH 5.0-6.5 (similar to neutrophil cytosol environments). FIG. 4A shows absorption spectra measured using a UV-Vis spectrometer for each type of nanoparticle supernatants collected 2 h after incubation. Absorption of DOX molecules at 480 nm was observed when DOX-hyd-BSA NPs were incubated at pH 5.0 and 6.5, and the absorption was dependent on pH, indicating that DOX was able to be cleaved from BSA NPs. However, we did not observe DOX in the supernatant of DOX-hyd-BSA NPs at pH 7.4, suggesting that DOX could be tightly trapped in NPs in the physiological condition. DOX-ab-BSA NPs did not show the absorption of DOX in their supernatants from pH 5.0 to 7.4 because the bond between DOX and BSA was not pH-responsive. A time course of drug release was also measured in DOX-BSA NPs and their pH dependence in vitro, as shown in FIG. 4B. DOX-ab-BSA NPs released 10-20% DOX at pH 5.0-7.4 in 48 h. In contrast, DOX release from DOX-hyd-BSA NPs was rapid in first 10 h (e.g. 60% DOX release at pH 6.5 and 80% at pH 5.0), indicating that acidic environments in a cell may trigger DOX release after DOX-hyd-BSA NPs were internalized.

Whether DOX can be released from NPs to promote cell death was thereafter determined. HL60 cells, as known in the art, was used because they are neutrophil-like after their differentiation. FIG. 4C shows that the cytotoxicity was similar for both free DOX and DOX-hyd-BSA NPs, and their cytotoxicity was more efficient compared to that of DOX-ab-BSA NPs, as shown in FIG. 4D. The result empirically showed that DOX can be released from BSA NPs via the cleavage of hydrazone bonds between DOX and BSA to cause cell death. Furthermore, the data shown in FIG. 4D illustrates that BSA NPs alone did not have any toxicity to cells in a wide range of concentrations (0-400 μg/ml) and BSA NPs were used at 100 μg/ml per mouse in animal experiments of the present application.

Neutrophil activation is required for tissue infiltration that contributes to inflammatory responses, so targeting activated neutrophils may increase drug delivery, avoiding systemic toxicity. In the present application, it was found that activated neutrophils can take up BSA NPs, but development of their responsive drug delivery systems to treat inflammatory disorders was unknown by those of ordinary skill in the art in this field. Furthermore, it is unclear whether inflammatory responses upregulate Fey receptors (a protein found on the surface of certain cells—including, neutrophils, macrophages, etc.) to mediate the uptake of BAS NPs. It was established herein that intravital microscopy of mouse cremaster venules enabled the study of this mechanism in a live animal (mouse). First, anti-CD16/32 (anti-Fcγ) was injected in a mouse via the tail vein, and 3 h later the cremaster tissue was exposed under an intravital microscope and stained neutrophils by intravenous (i.v.) injection of anti-mouse LY-6G antibody (a mouse neutrophil marker). Using such a methodology enabled the studying of the activities of resting (unstimulated) neutrophils in vivo.

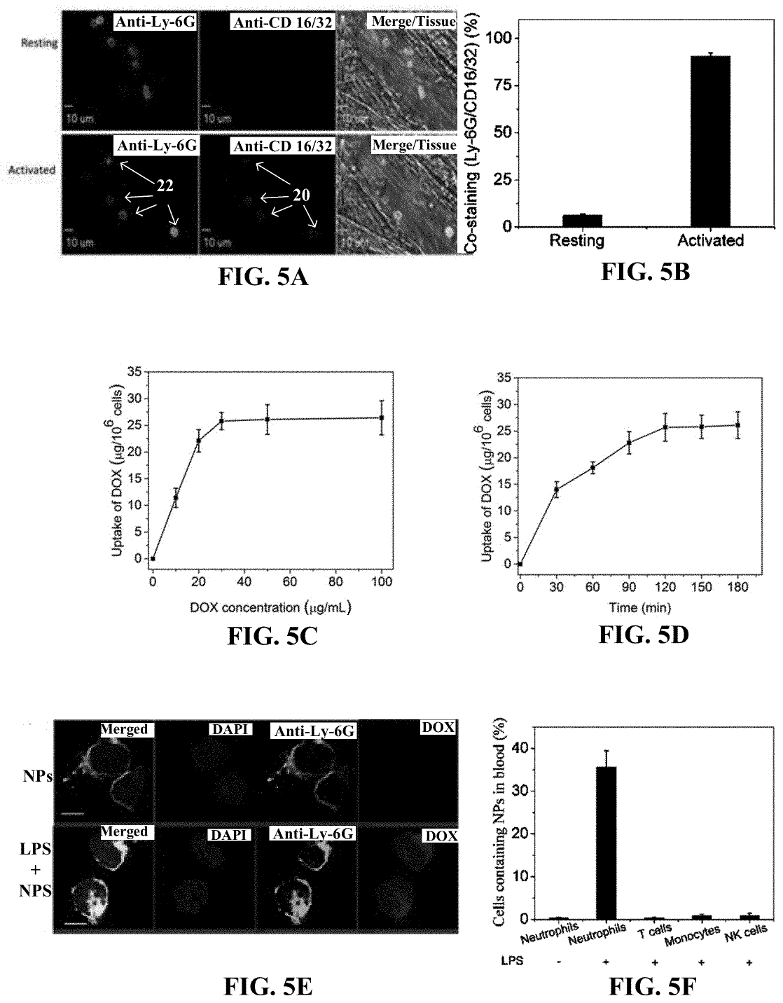

FIG. 5A are images that show neutrophil activation upregulating Fcγ receptors to mediate nanoparticle uptake. The images shown in FIG. 5A were taken using a Nikon A1R⁺ resonant-scanning confocal microscope at 488 nm and 640 nm. Scale bars, 10 μm. The resting condition of neutrophils, as shown in FIG. 5A, was established by no intrascrotal injection of TNF-α (0.5 μg per mouse) and the tail vein injection of Fcγ antibodies 3 h before performing intravital microscopy. The intravital images of FIG. 5A did not show staining of anti-CD16/32 (antibodies) on neutrophils, indicating that resting neutrophils did not highly express anti-CD16/32.

To activate neutrophils in vivo, the mouse cremaster tissue was challenged with intrascrotal (i.t.) injection of TNF-α 3 h before imaging. The bottom panel of FIG. 5A thus shows images of such intrascrotal injection of TNF-α (0.5 μg per mouse) activated neutrophils, wherein the images were obtained after the subject mouse was i.v. administered with anti-CD16/32 and anti-LY-6G antibodies. It is noted that Alexa Fluor 647-labeled anti-mouse CD16/32 (ref Char. 20) and Alexa Fluor 488-labeled anti-mouse LY-6G (ref Char. 22) antibodies were utilized to stain Fey receptors and neutrophils, respectively. Co-localization of anti-CD16/32 and anti-LY-6G was clearly observed (as shown in Merge/tissue image of Activated neutrophils), indicating that neutrophils were upregulated to express Fcγ receptors after the mouse was challenged by TNF-α.

FIG. 5B shows the percentage of co-staining between anti-mouse CD16/32 and anti-mouse LY-6G based on intravital images of FIG. 5A. Accordingly, FIG. 5B shows that 95% neutrophils expressed anti-CD16/32 after the stimulation by TNF-α, but it was 10% when neutrophils were inactivated. However, the 10% neutrophils may be associated with their activation induced by cremaster surgery for intravital imaging. The results are consistent with the conclusion that Fcγ receptors play a central role in mediating neutrophil uptake of BSA NPs. The results are consistent with the conclusion that Fcγ receptors play a central role in mediating neutrophil uptake of BSA NPs.

FIG. 5C and FIG. 5D show resultant uptake data of the present application, wherein DOX-hyd-BSA NPs by differentiated HL60 cells were evaluated in vitro. In particular, FIG. 5C shows uptake of DOX by differentiated HL60 cells after they (at 10⁶ cells/mL) were incubated with DOX-hyd-BSA NPs at different concentrations of DOX for 2 h. FIG. 5D shows time course of DOX uptake by differentiated HL60 cells after they (at 10⁶ cells/mL) were incubated with DOX-hyd-BSA NPs (at 30 μg/mL of DOX). Accordingly, DOX-hyd-BSA NPs is shown to be taken up efficiently by differentiated HL60 cells.

FIG. 5E shows confocal laser scanning microscopy (CLSM) images of blood neutrophils from healthy mice or LPS-challenged mice. 4 h after i.p. LPS injection, DOX-hyd-BSA NPs were i.v. administered to a mouse. 3 h later the mouse blood was collected, and neutrophils were isolated using anti-mouse LY-6G beads. Alexa Fluor 488-labeled anti-mouse LY-6G antibody was used to label neutrophils. Scale bars, 10 μm. Thus, the uptake of BSA-based NPs by neutrophils in vivo was assessed in LPS-induced inflammation mouse model. DOX-hyd-BSA NPs were i.v. administered to a mouse 4 h after intraperitoneal (i.p.) injection of LPS (20 mg/kg) to induce systemic inflammation. Blood neutrophils were collected at 3 h post-injection of NPs followed by staining anti-mouse LY-6G antibodies for analysis of nanoparticle uptake using confocal microscopy and flow cytometry. Without LPS challenge, BSA NPs inside neutrophils were not observed, but neutrophil uptake was observed after LPS challenge (see FIG. 5E). Interestingly, DOX fluorescence diffused in a whole cell, indicating that DOX may be released from NPs because low pH (acidic environment, often a pH of 4.0 to 7.4, more often often a pH of about 4.0-6.5) inside neutrophils induced the cleavage of a pH-labile linker between DOX and albumin.

FIG. 5F shows uptake of BSA NPs by blood leukocytes analyzed by flow cytometry. Neutrophils, T cells, monocytes and natural killer (NK) cells were isolated from blood and stained by Alexa Fluor-647-labeled anti-mouse LY-6G, CD3, CD115 and CD335 antibodies, respectively. All data expressed as mean±s.d. (3 mice per group). Neutrophils, T cells, monocytes and natural killer (NK) cells were isolated from blood and stained by Alexa Fluor-647-labeled anti-mouse LY-6G, CD3, CD115 and CD335 antibodies, respectively. All data was expressed as mean±s.d. (3 mice per group).

The flow cytometry result of FIG. 5F thus clearly demonstrated that neutrophil activation (challenged with LPS) was required for nanoparticle uptake. The uptake of DOX-hyd-BSA NPs was also analyzed in other immune cells in blood (again see FIG. 5F) and the results showed that T cells (anti-mouse CD3 antibody to mark T cells), monocytes (anti-mouse CD115 antibody to mark monocytes) and natural killer (NK) cells (anti-mouse CD335 antibody) to mark NK cells did not take up BSA NPs because of delayed activation of these adaptive immune cells. Taken together, BSA NPs can specifically target activated neutrophils for intracellular drug delivery during inflammation.

DOX is commonly used in cancer therapy but is known to cause inflammation at given dose levels in the art when used for cancer treatment. DOX induces cell death, but it is unknown whether the death of neutrophils is associated with apoptotic pathways. To address this question, the Applicants differentiated HL-60 cells because they are neutrophil-like cells. Phosphatidylserine on the outer leaflet of plasma membrane is a biomarker for cell apoptosis. Annexin V is commonly used to detect apoptotic cells by its binding to phosphatidylserine. 7-aminoactinomycin D (7AAD) is a fluorescent dye that is a membrane impermeant agent to identify dead cells.

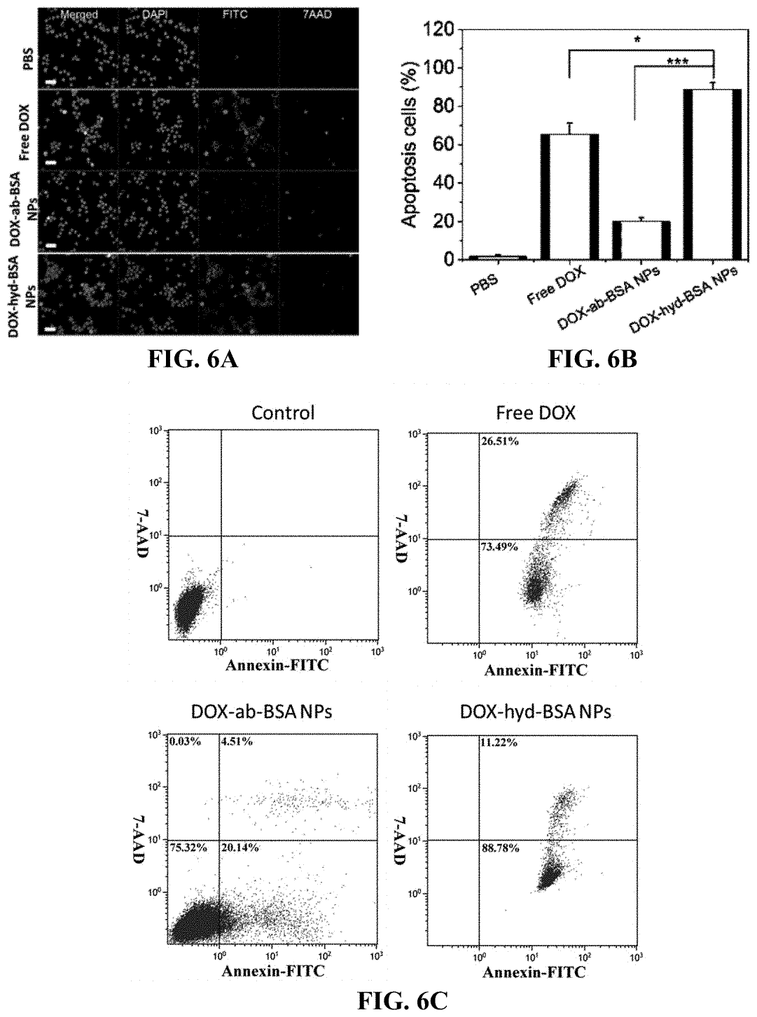

FIG. 6A shows confocal fluorescence microscopy (CLSM) images which indicate the apoptosis of differentiated HL-60 cells. Differentiated HL-60 cells were stained with Annexin V-FITC and 7AAD after the cells were treated with, among others, PBS, free DOX, DOX-ab-BSA NPs or DOX-hyd-BSA NPs for 24 h, respectively. Annexin V-FITC is an apoptosis marker and 7AAD (emission at 650 nm) is a fluorescent dye to mark dead cells. The confocal fluorescence microscopy showed that cells treated with free DOX or DOX-hyd-BSA NPs contained staining of Annexin-V-FITC, and less staining of 7AAD indicated that the cells were alive. In contrast, DOX-ab-BSA NPs did not induce cell apoptosis because DOX cannot be released from NPs. FIG. 6B and FIG. 6C show percentage of apoptotic cells analyzed by flow cytometry after HL-60 cells treated with various DOX formulations. Specifically, FIG. 6B shows percentage of apoptotic cells analyzed by flow cytometry after HL-60 cells treated with various DOX formulations and FIG. 6C shows flow cytometric analysis on apoptosis of differentiated HL-60 cells 24 h after the treatments with free DOX (0.2 mg/kg), DOX-ab-BSA or DOX-hyd-BSA NPs (equal to DOX of 0.2 mg/kg), respectively.

The flow cytometry analysis revealed that free DOX and DOX-hyd-BSA NPs caused cell apoptosis at 73% and 89%, respectively, whereas there were only 20% apoptotic cells after treatment with DOX-ab-BSA NPs. Furthermore, TUNEL (Terminal deoxynucleotidyl transferase (TdT) dUTP Nick-end Labeling), an assay to detect apoptotic cells, was used to confirm DOX-induced cell apoptosis.

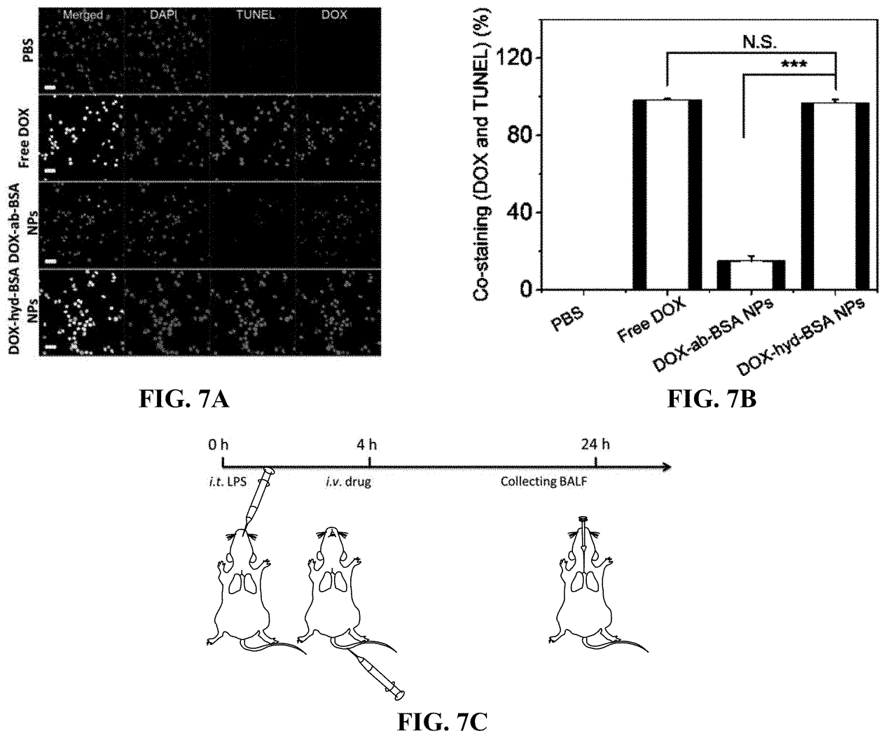

FIG. 7A images indicate that nearly 100% cells showed co-staining of DOX and TUNEL after the cells were treated with free DOX, suggesting that DOX effectively promoted cell apoptosis. When DOX-hyd-BSA NPs were used, co-staining of DOX and TUNEL was similar to that of free DOX, indicating that DOX can be released from NPs for intracellular delivery in neutrophils. In contrast, DOX-ab-BSA NPs treatment showed DOX signal in cells, but a few cells possessed TUNEL signal, suggesting that cell uptake of DOX-ab-BSA NPs did not mediate cell apoptosis. FIG. 7B shows percentage of co-staining between DOX and TUNEL based on CLSM images of FIG. 7A. Quantification, as shown in FIG. 7B, of co-staining of DOX and TUNEL in cells indicates that DOX-hyd-BSA NPs can effectively induce neutrophil apoptosis.

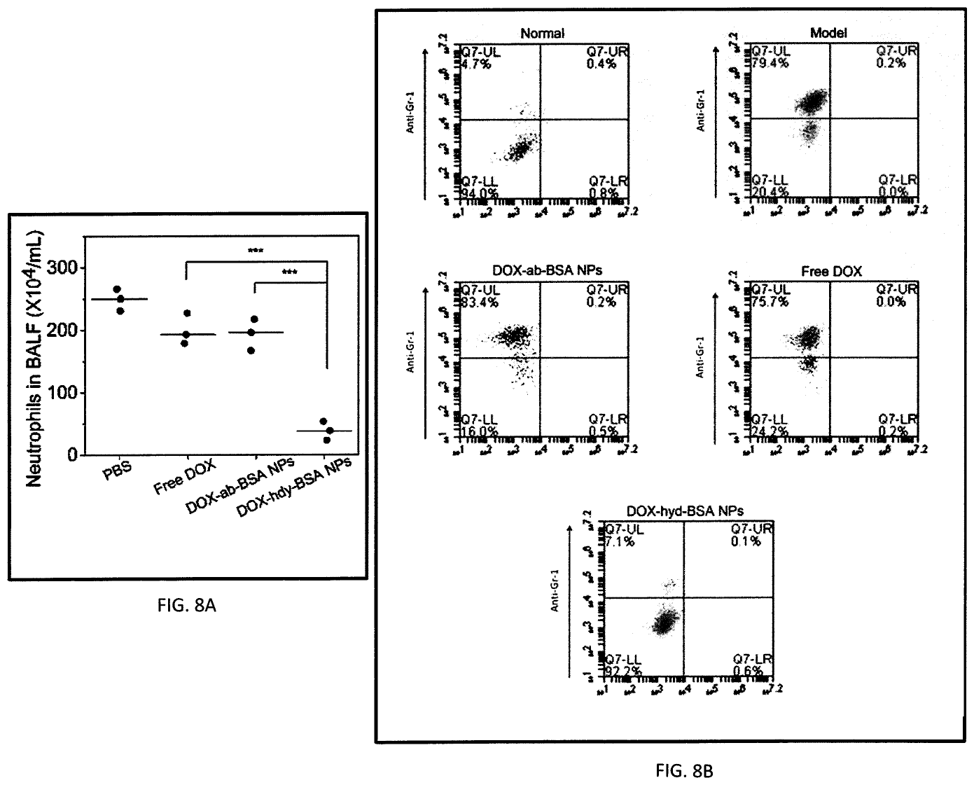

Acute lung inflammation is induced by LPS in bacterial infections and is associated with neutrophil recruitment to the lung. Here, it was determined whether targeted delivery of DOX to neutrophils in vivo could diminish neutrophil infiltration and related inflammatory responses in the mouse lung. FIG. 7C, which is related to the data shown in FIG. 8A and FIG. 8B, shows a graphical diagram of the experimental protocol in acute lung inflammation mouse model. To explain, intratracheal administration of LPS causes local lung acute inflammation, and subsequently neutrophils transmigrate from blood to airspace in the lung. 24 h after i.v. injection of several DOX formulations, BALF was collected to assess neutrophil number and cytokines. Specifically, 4 h after intratracheal (i.t.) LPS challenge to the mouse lung, the mouse was i.v. injected with various DOX formulations or PBS. Bronchoalveolar lavage fluid (BALF) was collected for assessment of neutrophil infiltration from circulation to the lung airspace and cytokines.

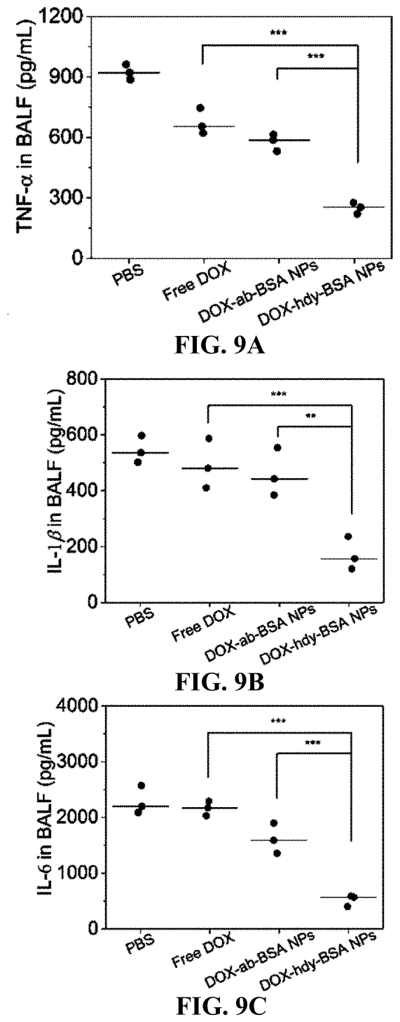

Accordingly, neutrophils were measured by flow cytometry, as shown in FIG. 8A and FIG. 8B. The results shown in FIG. 8A and FIG. 8B illustrate that after the treatment with DOX-hyd-BSA NPs at a very low dose of, for example 0.2 mg/kg (DOX), neutrophils were surprisingly and dramatically shown to decrease compared to controls (free DOX- and DOX-ab-BSA NPs-treated groups), suggesting that DOX-hyd-BSA NPs effectively inhibited the trafficking of activated neutrophils to the lungs in vivo because neutrophil death was induced by DOX. Lung neutrophil infiltration could enhance inflammatory responses to produce several inflammatory factors in BALF (such as TNF-α, IL-1β and IL-6). Subsequently, inflammatory factors were measured wherein it was found that these cytokines (TNF-α, IL-1β and IL-6) remarkably and also surprisingly, decreased after treatment with DOX-hyd-BSA NPs compared to other treatments (such as, free DOX and DOX-ab-BSA NPs) (see FIG. 9 A, FIG. 9B, and FIG. 9C). The results indicated that reduced cytokines may be associated with diminished neutrophil recruitment after neutrophil apoptosis.

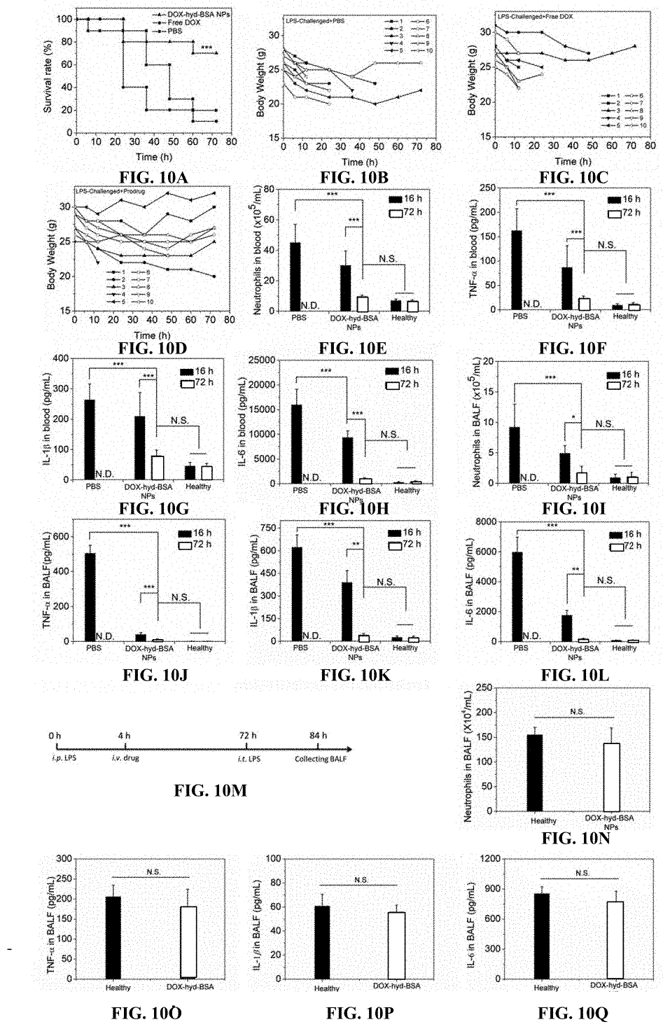

Sepsis, characterized as systemic inflammatory response syndrome (SIRS), is a life-threatening organ dysfunction caused by a dysregulated host response to infections. Currently, the supportive care is a primary option, and no pharmacological therapies are available in clinic. The early stage of sepsis is strongly correlated to neutrophil tissue infiltration and related inflammatory responses. In an endotoxin-induced sepsis mouse model, i.p. injection of LPS (50 mg/kg) caused acute and serious systemic inflammation, resulting in mouse death in a short period. We i.v. administered free DOX or DOX-hyd-BSA NPs 4 h after LPS challenge. Free DOX showed the similar death rate in 72 h to the PBS treatment, indicating that the treatment with free DOX did not protect mouse death from sepsis (FIG. 10A). By comparison, 70% mice survived in 72 h when mice were treated with DOX-hyd-BSA NPs, showing the benefit of the NPs to sepsis. During experiments, the mouse weight changes associated with metabolism and diet were monitored (FIG. 10B, FIG. 10C, and FIG. 10D). An interesting feature was found, in which dead mice dramatically lost their weight. When mice survived, the initial weight loss was recovered and became stable. This phenomenon raised an interesting question how mice overcome sepsis.

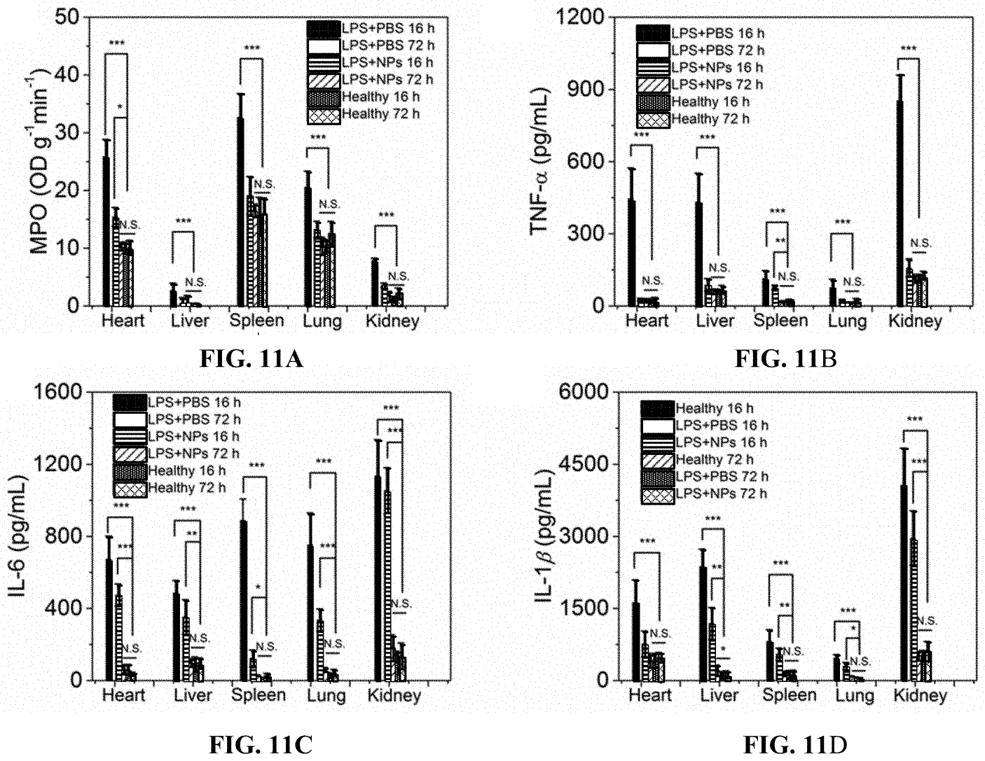

To address this question, a time course of neutrophil numbers and cytokines in peripheral blood (FIG. 10E, FIG. 10F, FIG. 10G, and FIG. H) and lung BALF were systemically investigated (FIG. 10I, FIG. 10J, FIG. 10K, and FIG. 1). Observed neutrophils dramatically decreased in blood and lung BALF in the first 16 h after LPS challenge, and cytokines also decreased accordingly when DOX-hyd-BSA NPs were administered compared to PBS treatment. The results suggest that DOX-hyd-BSA NPs may mediate apoptosis of inflammatory neutrophils in blood responsible for inhibition of neutrophil recruitment to the mouse lungs. Neutrophil death induced by DOX finally inhibited the systemic inflammatory responses (FIG. 10M, FIG. 10N, FIG. 10O, FIG. 10P, and FIG. 10Q). Most importantly, neutrophil number and cytokine contents at 72 h in mice treated with DOX-hyd-BSA NPs resumed to their levels as in healthy mice. The result indicates that DOX-hyd-BSA NPs do not inhibit the neutrophil production and their function in the bone marrow Inflammatory responses in other organs in sepsis (FIG. 11A, FIG. 11B, FIG. 11C, and FIG. 11D) were also checked. In particular, FIGS. 11A-D show DOX-hyd-BSA NPs decrease in systemic inflammation in the mouse sepsis model. More specifically, 4 h after (i.p.) LPS-challenge (50 mg/kg), the mice were treated with PBS or DOX-hyd-BSA NPs to show MPO activity (see FIG. 11A), TNF-α activity (see FIG. 11B), IL-6 activity (see FIG. 11C) and IL-1β activity (see FIG. 11D). The major organs were measured at 16 h and 72 h post-LPS challenge. All data expressed as mean±s.d. (5 mice per group at 16 h were used in the experiment, but 4 mice in NPs-treated group were used and all mice in PBS-treated group died at 72 h). Lung tissues were assessed after removal of BALF. * p value <0.05, ** p value <0.01, ***p value <0.001. The finding was that DOX-hyd-BSA NPs also significantly decreased inflammation in other organs. The result shows that targeting proinflammatory neutrophils by DOX-hyd-BSA NPs can inhibit systemic inflammation in sepsis.

Whether the treatment with DOX-conjugated BSA NPs impeded the innate immune responses of neutrophils when the second infection occurs was then addressed. The combinatory experiments were designed in which a mouse was i.p. challenged with LPS to cause sepsis, and then a mouse was treated with DOX-loaded NPs 4 h after the LPS challenge. At 72 h after the LPS challenge, the survival mouse was i.t. administered to examine whether neutrophils can transmigrate and respond to the second hit of LPS (see FIG. 10M of experimental protocol diagram).

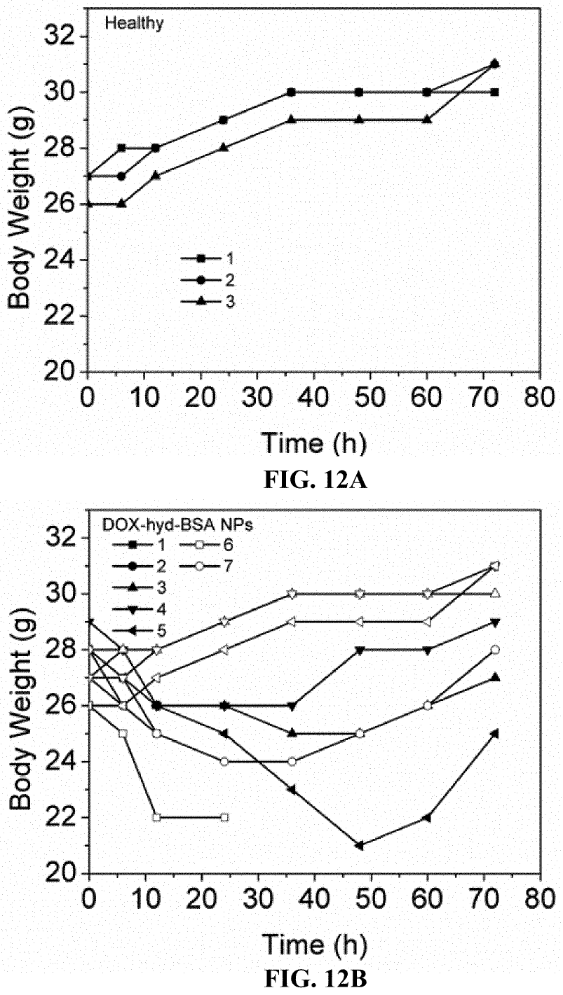

In the control, i.t. challenged healthy mice were challenged with LPS. During the experiments, mouse weight was monitored, as shown in FIG. 12A and FIG. 12B. FIG. 12A thus shows mouse body weights of three Mice as the control group (healthy mice) and FIG. 12 B shows seven mice of the DOX-hyd-BSA NPs-treatment group (using a 0.2 mg (DOX)/kg dose) (both the control group and treatment group monitored using the graphical experiment protocol shown in FIG. 10M). The finding was that the survived mice in sepsis after the treatment with DOX-conjugated NPs returned to their initial weight, indicating the therapeutic effect of NPs.

Innate immune responses were investigated of neutrophils using an acute lung inflammation model since this model allowed to measure neutrophil mobility in vivo. The results (see FIG. 10N, FIG. 10O, FIG. 10P, and FIG. 10Q) showed that the mice treated with pH-responsive DOX-conjugated NPs in sepsis can immediately respond to the challenge of LPS like what the healthy mice did. The data are evident to support that DOX-conjugated NPs specifically regulate proinflammatory neutrophils, but do not interfere with the production of new neutrophils in the bone marrow, thus avoiding the systemic immune suppression.

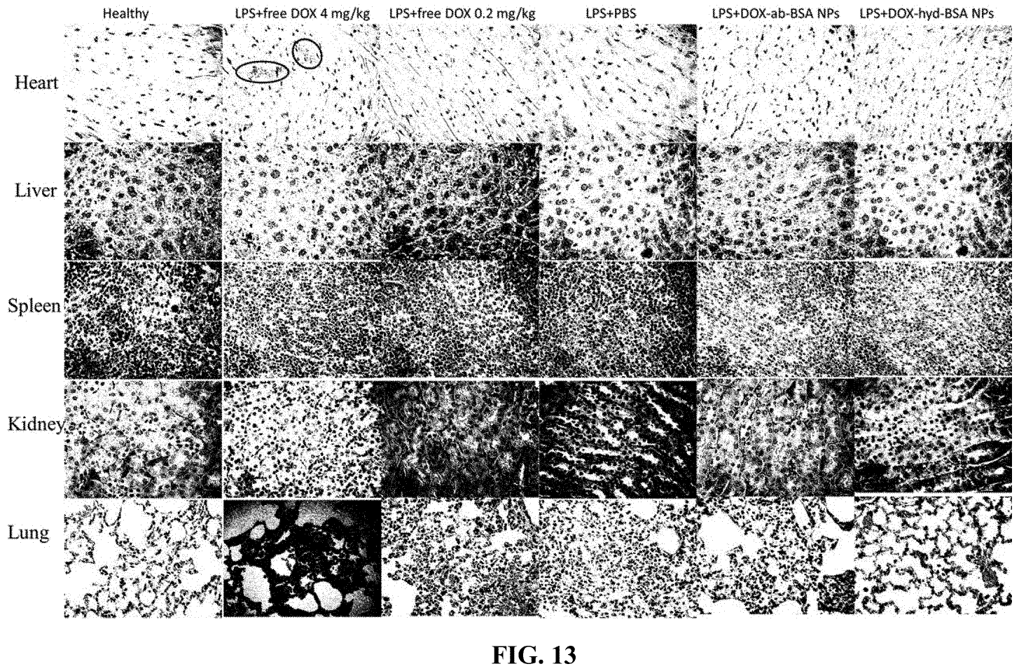

It is known that DOX shows severe cardiac toxicity in cancer therapy, so DOX-conjugated BSA NPs was addressed whether such NPs caused a side effect at a dose of 0.2 mg/kg used in anti-inflammatory therapies, as disclosed herein. Following the protocol as shown in FIG. 7C, the histology of several organs (heart, liver, spleen, lung, and kidney) was studied after administration of free DOX and several BSA-based NPs (see FIG. 13). Specifically, FIG. 13 shows Toxicity of DOX-conjugated BSA NPs evaluated by histological analysis. H&E-stained sections of major organs of healthy and LPS-challenged mice after treatments with PBS, free DOX (4 or 0.2 mg/kg), DOX-ab-BSA NPs, or DOX-hyd-BSA NPs (equal to 0.2 mg/kg at DOX). Scale bar, 20 μm. It was observed that DOX-conjugated NPs and free DOX (0.2 mg/kg DOX) did not show any noticeable signs of tissue or cellular damage. In contrast, the high dose of DOX (4.0 mg/kg) induced myocardial damage, as shown by vacuolization and myofibril loss highlighted by the circles. Furthermore, the damage of liver, spleen, kidney and lung by DOX in 0.2-4.0 mg/kg was not observed.

It is important to note that no toxicity was found in the heart, liver, spleen, lung and kidney at 0.2 mg/kg DOX either in free DOX or in BSA NPs formulations. However, at the high dose of free DOX treatment (4 mg/kg), the myocardial damage was apparent because of intensive vacuolization and myofibril loss. The result indicated that the low dose regime of DOX used in our anti-inflammatory therapy surprisingly is tolerant and there is no apparent organ toxicity the mouse experiments of the present invention.

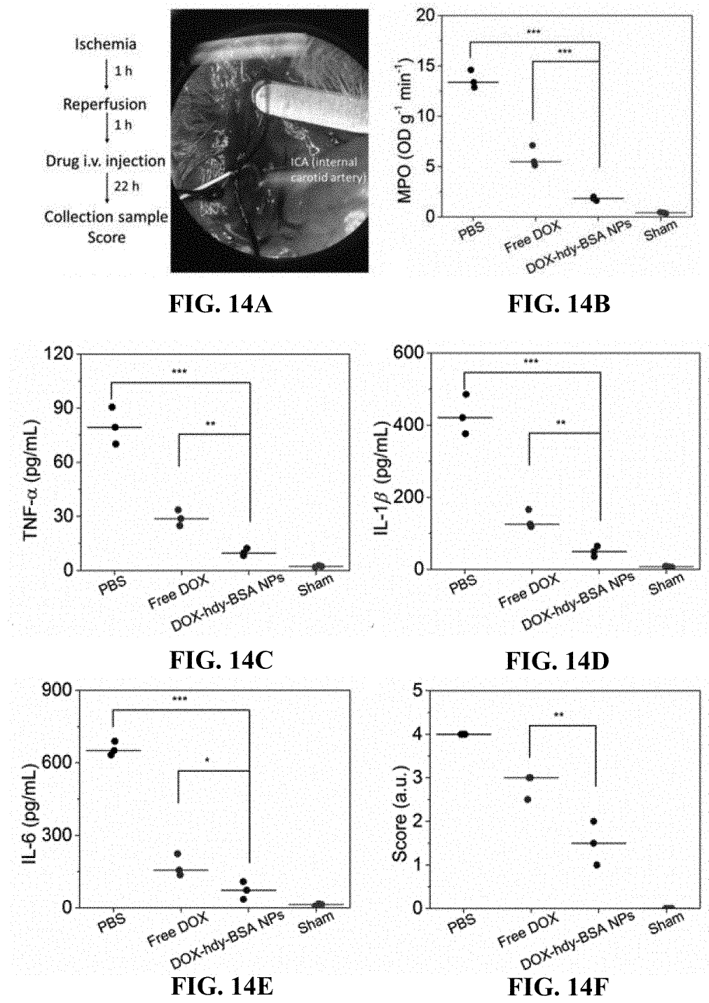

FIGS. 14A-F show DOX-hyd-BSA NPs mitigation of neutrophil-induced neuroinflammation in cerebral I/R mouse model to restore neurological functions. FIG. 14A specifically shows the experimental design to examine the benefit of DOX-hyd-BSA NPs in cerebral I/R. FIG. 14B shows MPO activity, FIG. 14C shows TNF-α activity, FIG. 14D shows IL-1β activity, and FIG. 14E shows IL-6 activity in brain damaged tissues at 22 h post administration of PBS, free DOX, and DOX-hyd-BSA NPs. FIG. 14F shows Mouse neurological behavior scores after treatments with PBS, free DOX, and DOX-hyd-BSA NPs at 0.2 mg/kg of DOX. All data expressed as mean±s.d. (3 mice per group). Statistics was performed by a two-sample Student's t test (*p<0.05, **p<0.01, ***p<0.001).

Stroke is a major cause of death and adult disability. Most strokes are related to ischemia that is blood vessel clogs in the brain. Currently, reperfusion is a surgery option to restore blood circulation, but reperfusion causes a secondary tissue damage due to neuroinflammation. Neutrophils play a central role in this neuroinflammation, such as in cerebral ischemia/reperfusion (I/R). DOX-hyd-BSA NPs was examined as to whether it can benefit the therapy for ischemic stroke. In experiments, a middle cerebral artery occlusion (MCAO) mouse model was established to mimic cerebral ischemia/reperfusion (see FIG. 14A). 1 h after inclusion with a filament in internal carotid artery (ICA), reperfusion was performed by withdrawal of the filament. 1 h after perfusion, NPs were i.v. administered, and brain tissues were collected for analysis of neutrophils and cytokine levels at 22 h post-injection of free DOX or NPs (see FIG. 14 B, FIG. 14C, FIG. 14 D, and FIG. 14 E). Compared to free DOX and PBS treatments, it was observed that DOX-hyd-BSA NPs significantly reduced neutrophil numbers (MPO (myeloperoxidase), a marker of neutrophils)) and cytokines in the brain, implying that inhibition of neutrophils by DOX-hyd-BSA NPs is useful to prevent the secondary brain damage from inflammatory responses of neutrophils induced by the surgery for ischemic stroke therapy. Neurological deficits related to cerebral I/R were evaluated after the treatment of DOX-hyd-BSA NPs (see FIG. 14F), indicating that DOX-hyd-BSA NPs enhanced mouse neurological recovery and improved mouse movements compared to several controls.

### Discussion

Inflammation associated with the innate and adaptive immune systems is a defense to infections or tissue injury. However, when unchecked, inflammation may cause autoimmune or inflammatory disorders, such as sepsis, stroke, aging and even cancer. Anti-inflammatory agents have been developed to inhibit inflammation pathways, such as NF-κB pathway, but their off-targeting delivery causes systemic toxicity. In addition, anti-cytokine therapies have been used in clinic to neutralize cytokine storm during inflammatory responses. The cytokines, such as TNF-α and IL-1β, are mediators of diseases, thus they are the targets for anti-cytokine therapy using anti-TNF-α and anti-IL-1β. Although blocking cytokines may reduce inflammation, it renders the host susceptible to infections. Selective targeting to immune cells for intracellular drug delivery may be a potential strategy to manage inflammatory responses to infections or tissue injury to maintain immune homeostasis.

Apoptosis is a natural process of cell death to maintain the body homeostasis. For example, neutrophils have a short lifespan and it is regulated by apoptosis to preserve constant numbers of neutrophils in circulation to warrant the immune homeostasis, protecting the host damage from neutrophils. Inspired by this natural neutrophil apoptosis, a means disclosed herein is used to specifically target inflammatory neutrophils using NPs that deliver doxorubicin to promote neutrophil apoptosis to treat inflammatory responses resultant from, but not limited to: inflammatory disorders, immune disorders, infections, etc. (see FIG. 1A and FIG. 1B).

Example inflammatory responses can include at least one response due to a disorder or disease or infection selected from: a stroke, an aging disorder, cancer, Huntington's disease, encephalitis, autoimmune disorders, immune-complex vasculitis, lupus, cardiomyopathy, ischemic heart disease, atherosclerosis, chronic liver failure, brain and spinal cord trauma, sarcoidosis, arthritis, rheumatoid arthritis, inflammatory bowel disease, ileitis, ulcerative colitis, Barrett's syndrome, Crohn's disease, asthma, onchocerciasis, uveitis, sympathetic ophthalmitis, periodontitis, tuberculosis, glomerulonephritis, nephrosis, sclerodermatitis, psoriasis, eczema, multiple sclerosis, AIDS-related neurodegeneration, Alzheimer's disease, meningitis, encephalomyelitis, Parkinson's disease, acute lung inflammation/injury, and sepsis.

Such an approach enhances inflammation resolution without suppression of the host immune system. Accordingly, a DOX-conjugated BSA NPs was developed, as disclosed herein, that specifically binds to activated neutrophils and controls DOX release when NPs are taken up by neutrophils (e.g., see FIG. 3A-F and FIGS. 5A-F). Most importantly, it has been demonstrated that the uptake of NPs is required when neutrophils are activated during inflammation. Furthermore, the uptake of NPs is dependent on Fcγ receptors expressed on neutrophils as shown by intravital microscopy, as discussed above. This was supported by results herein that the uptake of BSA NPs dramatically decreased in Fcγ receptors-knockout mice. This is consistent with the function of Fey receptors to endocytose denatured BSA induced by the formation of NPs, but not BSA molecules. However, other immune cells (such as monocytes, T cells and NK cells) do not take up NPs in LPS-induced acute inflammation, and this may be associated with their adaptive characteristics. DOX has been studied to treat cancer because it can cause cell death. High doses of DOX administration in cancer therapy, as briefly discussed above, causes heart toxicity and can lead to systemic inflammation. A key beneficial aspect of the present invention was that surprisingly, the low dose of DOX (0.1 mg/kg to 10 mg/kg) administered to a mouse did not cause inflammation and heart toxicity (see FIG. 13). This is also consistent with the tolerance of DOX used in cancer therapy. In an acute lung inflammation mouse model, administration of DOX-hyd BSA NPs inhibited neutrophil transmigration into the mouse lung, and also decreased cytokine levels compared to free DOX or PBS. The results clearly indicate that neutrophil apoptosis prevents trafficking of neutrophils in response to LPS-induced lung inflammation.

It had been demonstrated herein that DOX-hyd-BSA NPs can mediate the apoptosis of proinflammatory neutrophils to increase inflammation resolution, thus preventing acute lung inflammation/injury. Exaggerated neutrophil activation contributes to pathogenesis of sepsis and ischemic stroke, so the usefulness of DOX-conjugated NPs was examined to treat the example two diseases. In the LPS-induced sepsis mouse model, administration of DOX-hyd-BSA NPs increased the mouse survival to 70% vs 10-20% for controls (free DOX and PBS)). The present application shows that neutrophil apoptosis decreased neutrophil numbers in circulation and in the lungs, thus inhibiting neutrophil trafficking to mitigate systemic inflammation.

Most importantly, it was discovered that administration of DOX-hyd-BSA NPs did not impair neutrophil production in the bone marrow when neutrophil counts was compared in healthy mice. In addition, the mice survived from sepsis after the treatment with DOX-conjugated NPs can normally respond to the second hit of LPS like what heathy mice do. This interesting and unexpected and surprising result demonstrates a new concept to treat inflammatory diseases by specifically targeted delivery of therapeutics to proinflammatory neutrophils.

Such a novel approach of the present invention avoids the systemic suppression caused by currently used anti-inflammatory agents. In the ischemic stroke mouse model, it has been shown herein have that inhibition of neutrophil trafficking by DOX-hyd-BSA NPs rescued mouse neurological damage during reperfusion therapy to ischemic stroke. Collectively, the methods herein reveal a new concept to develop anti-inflammatory therapies by targeting immune cell apoptosis pathways using NPs.

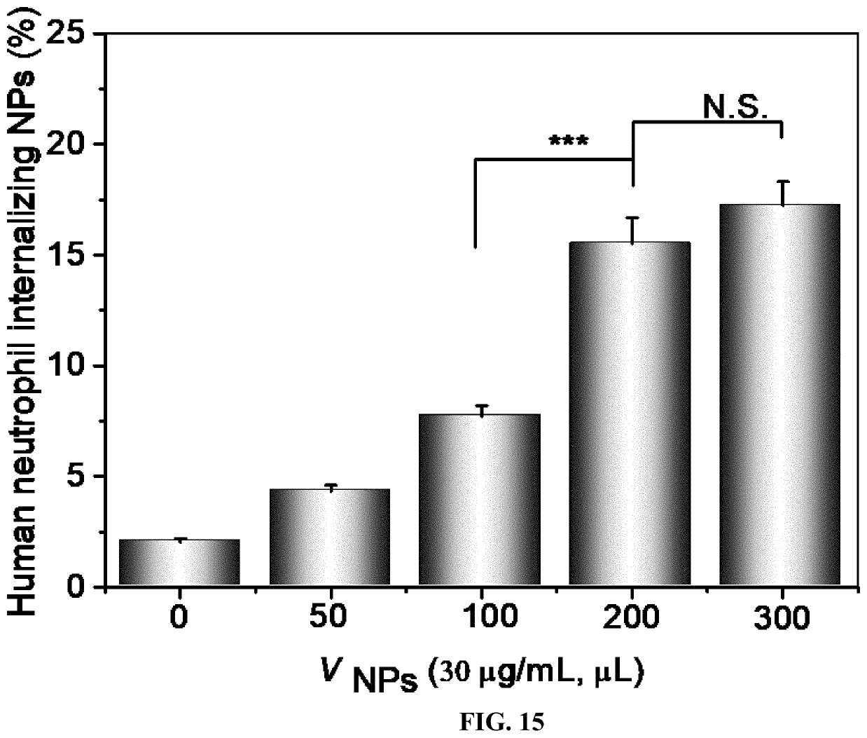

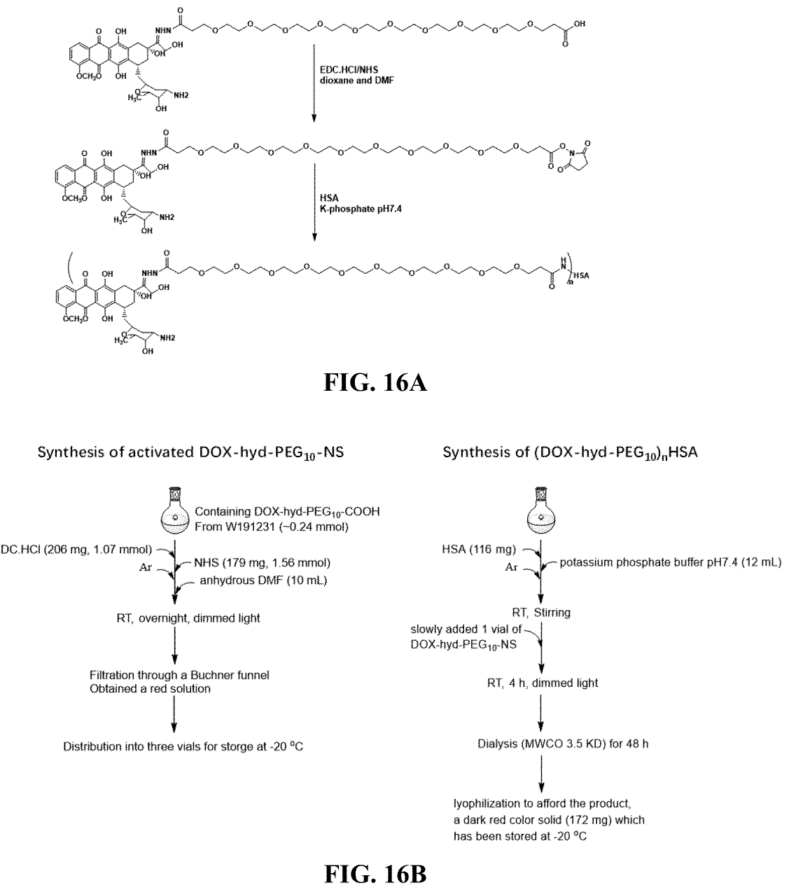

It is also to be noted that the methods herein also pertain to making human albumin nanoparticles similar to BSA NPs. FIG. 15 shows this ability by the data of uptake of DOX-hdy-BSA NPs by human neutrophils. Human neutrophils were incubated with DOX-hyd-BSA NPs (the concentration at 30 ug/ml) for 2 h, and then the cells were washed and analyzed by flowcytometry. FIG. 16 A and FIG. 16 B shows the synthesis of human serum albumin (HAS) conjugated with DOX. Specifically, FIG. 16A shows the molecular structure of DOX and a linker between DOX and human serum albumin while FIG. 16A shows the detailed synthesis of DOX-hyd-HSA Thus, approaches of the present invention for BSA NPs provides for translational possibilities using, for example, glutaraldehyde or other biocompatible coupling agents.

### Materials and Methods

### Materials

Bovine serum albumin (BSA), triethylamine (TEA, 99%), 1-ethyl-3-(3-(dimethylamino)propyl) carbodimide (EDC), N-hydroxysuccinimide (NHS), lipopolysaccharide (LPS, Escherichia coli 0111: B4), formaldehyde solution and dimethyl sulfoxide (DMSO) were purchased from Sigma-Aldrich (St. Louis, Mo.). doxorubicin hydrochloride was purchased from Wuhan Yuan Cheng Gong Chuang Co. Dicarboxyl poly (ethylene glycol) (HOOC-PEG-COOH, M_{w}=600) was purchased from Creative PEGWorks. Glutaraldehyde was obtained from Electron Microscopy Sciences (Hatfield, Pa.). RPMI-1640 and other mediums were purchased from Lonza (Walkersville, Md.). HBSS buffer (without Ca²⁺, Mg²⁺ and phenol red) was obtained from Corning (Corning, N.Y.). Recombinant human and mouse TNF-α (carrier-free, purity >98%), Alexa Fluor-647 anti-mouse CD16/32, CD3, CD115 and CD335 antibodies, Alexa Fluor-488/647 anti-mouse Ly-6G antibody, Cell Meter™ TUNEL apoptosis assay kit and ELISA kits for TNF-α, IL-1β and IL-6 were purchased from Biolegend Inc. (San Diego, Calif.). Annexin V/dead cell apoptosis kit and 4′,6-diamidino-2-phenylindole (DAPI) were purchased from Invitrogen (Carlsbad, USA). Human HL-60 cell lines were obtained from ATCC (Manassas, Va.). Penicillin streptomycin (pen strep) and glutamine (100×) were purchased from Life Technologies (Grand Island, N.Y.). PierceTMBCA protein assay kit was purchased from Thermo Fisher Scientific. All other chemical and biological reagents were used as they received.

### Synthesis of DOX-Hyd-BSA

DOX-hyd-PEG-COOH was synthesized according to the previous study with some modifications and the synthetic route was discussed above. In brief, HOOC-PEG-COOH (2 mmol) was dissolved in DMSO at 1 mmol/mL and was activated by NHS/EDC (1:1 at the molar ratio) with the molar ratio of EDC to HOOC-PEG-COOH to be 3:1. Then NH₂NH₂.H₂O (1 eq relative to HOOC-PEG-COOH) and TEA were dropwise added into the solution. The reaction was carried out for 24 h with gently stirring at room temperature to obtain HOOC-PEG-CONHNH₂, followed by adding 2.0 mmol DOX and 10 mmol TFA into the solution. The resulting mixture was stirred for 48 h in dark at room temperature. DOX-hyd-PEG-COOH conjugate was received after dialysis in deionized water and frozen drying. Finally, DOX-hyd-PEG-COOH was conjugated to BSA through amino bonds. Briefly, 4.0 mmol DOX-hyd-PEG-COOH was firstly dissolved in 20 mL dioxane, and then NHS/EDC (4.0 mmol/4.0 mmol) and TEA (10 mmol) were added in the solution. The reaction was carried out for 12 h in dark at room temperature. Then 1.0 mmol BSA was dissolved in 20 mL deionized water and BSA solution was mixed with DOX-hyd-PEG-COOH. During the reaction process, the pH value of reaction mixture was maintained at 7 by dropwise adding 0.1 mmol/mL NaOH solution. The solution was stirred for 4 h, and then the pH was quickly adjusted to 9 by adding 0.1 mmol/mL NaOH solution. DOX-hyd-BSA powder was obtained after the solution was dialyzed in deionized water for 2 days followed by 2 days of lyophilization. DOX-ab-PEG was synthesized as a negative control by using HOOC-PEG-COOH to conjugate DOX instead NH₂NH₂.H₂O.

### Preparation of BSA NPs

BSA NPs were prepared by desolvation. DOX-hyd-BSA or DOX-ab-BSA was dissolved in deionized water at 2.5 mg/mL, but pure BSA NPs were formed at 20 mg/mL BSA solution. 30 min later, 1.2-3.5 mL ethanol was continuously added to the solution under stirring at 800 rpm at room temperature. 1 h later, stable BSA NPs were achieved by adding 20-80 μL of 2% glutaraldehyde to crosslink amine residues in BSA molecules. The resulting suspension was stirred overnight in dark at room temperature. Finally, the pellet of NPs was obtained after three times of the centrifugation at 20,000 g for 30 min at 4° C. After lyophilization, it was found that the particle formation efficiency was 70-80% for DOX-hyd-BSA and 80-90% for BSA respectively. The nanoparticle pellet was re-suspended in PBS or 5% glucose for the study.

### Characterization

NMR spectra measurements were executed on a Bruker AVANCE III 500 (Switzerland) spectrometer operating at 500 MHz, using deuterated chloroform (CDCl₃-d) or D₂O as solvent. To study the loading efficiency of DOX in BSA NPs, conjugated DOX was determined by UV-Vis spectroscopy. 1 mg DOX-hyd-BSA NPs were dissolved in 1 mL of 1 mol/L HCl and stirred for 3 h at 50° C. Afterwards, the solution was centrifuged at 20,000 g for 30 min at 4° C., and the supernatant was collected for DOX analysis at 480 nm. The drug conjugating content (DCC) was defined as the weight ratio of drug conjugated to BSA. The DCC of DOX-hyd-BSA or DOX-ab-BSA was 2.4% or 2.5%, respectively.

Particle size and polydispersity indexes (PDI) were measured using Malvern Zetasizer Nano90 (Westborough, Mass.). The samples were incubated in PBS at pH 7.4 for 2 h, and the measurements were conducted in a 1.0 mL quartz cuvette using a diode laser of 633 nm at 25° C. and a scattering angle was fixed at 90°. To evaluate their serum stability, nanoparticles were dispersed in 20% FBS PBS solution (pH 7.4) at a final concentration of 1 mg/mL. Nanoparticle sizes were measured at the predefined time points. Transmission electron microscopy (TEM) of nanoparticles was also performed using a FEI Technai G2 20 Twin TEM (Hillsboro, Oreg.).

To confirm the pH-triggered drug release property, BSA NPs, DOX-ab-BSA NPs and DOX-hyd-BSA NPs were incubated in PBS at pH 7.4, 6.5 and 5.0 for 2 h, respectively. After centrifugation, the supernatants were collected to determine DOX concentrations using a UV-Vis spectrometer.

### In Vitro Drug Release Profile

Release profiles of DOX from NPs were studied using dialysis method at 37° C.(33) Briefly, 3 mg of NPs was dispersed in 3 mL (Vₑ) PBS at different pH, and then was placed in a dialysis bag (MWCO 3500 Da). The dialysis bag was immersed in 47 mL PBS (pH 7.4, 6.5 or 5.0) in a beaker. The beaker was then placed in a 37° C. water bath and stirred at 110 rpm. The samples were drawn at desired time intervals and the drug concentration was measured using UV-Vis absorption. The experiments were carried out in triplicate at each pH value. The accumulative drug release percent (Eᵣ) was calculated based on equation (1).

$${E}_{r} ( \% ) = \frac{{V}_{e} {\sum }_{1}^{n - 1} {C}_{i} + {V}_{0} {C}_{n}}{{m}_{d r u g}} \times 100 \qquad ( 1 )$$

where, m_{drug} represents the amount of DOX in NPs, V₀ was the whole volume of release media (V₀=50 mL), Cᵢ represents the concentration of DOX in the ith sample.

### Cell Culture Condition L

HL-60 cells (a human promyelocytic leukemia cell line) were cultured in a RPMI1640 medium containing 10% fetal bovine serum (FBS), 100 units/mL streptomycin and 100 units/mL penicillin, and differentiated into PMN-like cells by adding 1.3% (v./v.) DMSO for 96 h as previously reported.(34, 35) Cells were maintained in an incubator in a humidified atmosphere containing 5% CO₂ at 37° C.

### In Vitro Cytotoxicity

Cytotoxic effects of BSA NPs on HL-60 cells were measured by CCK-8 assay. HL-60 cells were plated in 96-well plates (Costar, Corning, N.Y.) at 5,000-10,000 cells/well. After incubation for 24 h, the culture medium was removed and a complete medium with various concentrations of free DOX, BSA and DOX-conjugated BSA NPs was used to incubate cells for 24 h, respectively. 10 μL of the solution cell proliferation reagent (Promega, Madison, Wis.) per well was added. Then, the cell viability was measured by a Synergy Neo fluorescence plate reader (BioTek, Winooski, Vt.) at 490 nm.

### Apoptosis Analysis

To investigate the apoptosis of neutrophils induced by DOX, Annexin V-FITC and 7AAD were used to double stain differentiated HL-60 cells. The cells were seeded in a 6-well plate at a density of 1×10⁶ cells/well, and treated with free DOX, DOX-hyd-BSA NPs and DOX-ab-BSA NPs at a DOX concentration of 3 μg/mL for 24 h at 37° C. The cells were re-suspended in a binding buffer for staining of Annexin V-FITC and 7AAD (emission at 650 nm) according to the manufacturer's protocol (Invitrogen). Stained cells were analyzed using a flow cytometer and imaged by a Nikon A1R⁺ confocal laser scanning microscope.

To further confirm the apoptosis of differentiated HL-60 cells induced by DOX, TUNEL assay was also performed using Cell Meter™ TUNEL (green fluorescence) Apoptosis Assay Kit (AAT Bioquest, Inc.) according to the manufacturer's protocol and the images were taken by a Nikon A1R⁺ confocal laser scanning microscope.

### Mice

Adult CD-1 (male, 22-30 g, 4-6 weeks) were purchased from Harlan Laboratories (Madison, Wis.). The mice were maintained in polyethylene cages with stainless steel lids at 20° C. with a 12 h light/dark cycle and covered with a filter cap. Animals were fed with food and water ad libitum. All animal care and experimental protocols used in these studies were approved by the Washington State University Institutional Animal Care and Use Committee. All experiments were made under anesthesia using intraperitoneal (i.p.) injection of the mixture of ketamine (100 mg kg⁻¹) and xylazine (5 mg kg⁻¹) in saline.

### Nanoparticle Targeting to Neutrophils

To investigate whether DOX-hyd-BSA NPs can bind to activated neutrophils in vivo, the expression of Fcγ receptors on neutrophils using intravital microscopy was first studied. TNF-α (500 ng, 250 μL saline) was intrascrotally injected into a mouse (C57BL/6). At 3 h post-injection of TNF-α, the mouse was anesthetized with a mixture of ketamine and xylazine, as described above, and maintained at 37° C. on a thermo-controlled rodent blanket. A tracheal tube was inserted and a right jugular vein was cannulated for injection of antibodies Alexa Fluor-488-labeled anti-mouse LY-6G and Alexa Fluor-647-labeled anti-mouse CD16/32. A scrotum was incised, and the testicle and surrounding cremaster muscles were exteriorized onto an intravital microscopy tray. The cremaster preparation was perfused with thermo-controlled (37° C.) and aerated (95% N₂, 5% CO₂) bicarbonate-buffered saline throughout the experiment. Images were recorded using a Nikon A1R⁺ laser scanning confocal microscope with a resonant scanner. In studies on resting neutrophils in vivo, a mouse was not treated with TNF-α. 3 h after injection of Alexa Fluor-647-labeled anti-mouse CD16/32 antibody via tail vein, the mouse cremaster tissue was exposed for intravital imaging after intravenous (i.v.) administration of Alexa Fluor-488-labeled anti-mouse LY-6G antibody to stain neutrophils. The images were recorded using a Nikon A1R⁺ laser scanning confocal microscope with a resonant scanner.

To investigate cell uptake specificity of BSA NPs in circulation, blood cells were isolated and studied using confocal laser scanning microscopy (CLSM) and flow cytometry. Briefly, 4 h after LPS challenge (i.p., 20 mg kg⁻¹), DOX-hyd-BSA NPs were intravenously injected into mice. Healthy mice were used as control. 3 h later, the whole blood was harvested in a heparinized tube from the heart. Neutrophils in blood were isolated by Pluriselect anti-mouse LY-6G S-pluribeads according to the manufacturer's protocol (Pluriselect, Spring Valley, Calif.). The cells were fixed with 2 mL of 4% paraformaldehyde for 30 min and were stained with Alexa Fluor 488-labeled anti-mouse LY-6G antibody. A slide smear of cell solution was prepared by 7620 Cytopro Cytocentrifuge (ELITech, Princeton, N.J.). A drop of Prolong Gold Antifade reagent with DAPI (Invitrogen, Eugene, Oreg.) was added on the cells, and a coverslip was applied on the slide. 4 h later, the cells were observed using a Nikon A1R⁺ confocal laser scanning microscope.

Flow cytometry to quantify neutrophil uptake of nanoparticles was also utilized. Blood leukocytes were isolated to determine the specificity of uptake of NPs in blood. In brief, 3 mL Histopaque 10771 was carefully layered on top of 3 mL Histopaque 11191 in a 15 mL centrifuge tube. Mouse whole blood was decanted on the top followed by centrifugation at 890 g for 30 min at 22° C. with a gentle acceleration. Leukocytes were located between plasma and the Histopaque 10771 layer. The leukocyte suspension was collected, and dissolved in PBS without Ca²⁺ and Mg²⁺. After the resulting cell suspension was centrifuged at 870 g for 5 min at 4° C., T cells, monocytes, NK cells were labeled by Alexa Fluor 647-labeled anti-mouse CD 3, CD 115 and CD 335 antibodies, respectively, for flow cytometric analysis.

### Acute Lung Inflammation Mouse Model

Mice were anesthetized, and placed in a supine position head up on a board tilted at 15°. Afterwards, 10 mg/kg of LPS in HBSS was intratracheally (i.t.) administrated to mice with a FMJ-250 High Pressure Syringe (Penn-Century, Wyndmoor, Pa.). Mice were held upright for 2 min after administration.

### Bronchoalveolar Lavage Fluid (BALF) Collection and Cell Counts

Mice were challenged with i.t. injection of LPS (10 mg/kg). 4 h later, PBS, free DOX (0.2 mg/kg or 4 mg/kg), DOX-ab-BSA NPs or DOX-hyd-BSA NPs (equal to 0.2 mg/kg of DOX) were i.v. injected. At 20 h post-drug administration, the mice were anesthetized with i.v. injection of a mixture of ketamine and xylazine. The BALF was collected by inserting a needle into the upper trachea. 0.9 mL HBSS was infused into the lungs and carefully withdrawn to obtain BALF. This process was repeated three times. BALF was collected and centrifuged at 350 g for 10 min at 4° C. The supernatant was collected for ELISA analysis. Afterwards, the cells were re-suspended in 0.5 ml of HBSS. The total cell number was determined with a hemocytometer.

Flow cytometry was utilized to quantify neutrophils in BALF. In detail, neutrophils from BALF were washed with 1 mL of 5% BSA in HBSS and centrifuged at 350 g for 10 min at 4° C. for three times, which was finally re-suspended in 400 μL of 5% BSA in HBSS. 3 μL of Alexa Fluor-647-labeled anti-mouse LY-6G antibody was added and incubated for 20 min in the dark, followed by washing with 1 mL of 0.1% BSA in HBSS under centrifugation for three times. Samples were then re-suspended in 400 μL 0.1% BSA in HBSS and filtered by a 100 μm filter, and analyzed by flow cytometer (Accuri C6 flow cytometer, BD Biosciences, San Jose, Calif.).

### Cytokines

BALF was collected and centrifuged at 350 g for 10 min as described above. Supernatants from BALF were harvested for ELISA analysis. Concentrations of TNF-α, IL-6 and IL-1β in supernatants were determined with commercial ELISA kits according to the manufacturer's instructions (Biolegend, San Diego, Calif.). The triplicate experiment was conducted.

### H &E Staining

After different treatments (healthy, PBS, 4 mg/kg of free DOX, 0.2 mg/kg of free DOX, DOX-hyd-BSA NPs and DOX-ab-BSA NPs, equal to 0.2 mg/kg of DOX for NPs), mice were sacrificed by carbon dioxide asphyxiation. Organs were removed and fixed with 10% formalin, embedded in paraffin, sectioned at 5 μm and stained with hematoxylin and eosin for pathology (RTPH 360 Rapid Tissue Processor Operator Manual and SS-2030 Linear Slide Stainer, General Data, and Leica RM 2145, Leica Microsystems). The samples were imaged by a microscope (ZEISS, Observer. Z1, USA).

### Survival Study in Sepsis Mouse Model

Adult CD-1 mice were i.p. injected with LPS (50 mg/kg) in the mouse sepsis model. 4 hours later, the LPS-challenged mice were grouped randomly (10 mice per group) and treated (i.v.) with PBS, free DOX (0.2 mg/kg), and prodrug DOX-hyd-BSA NPs (equal to 0.2 mg/kg of free DOX). The animals were monitored every 6 h in the first 12 h followed by monitoring mice every 12 h in 72 h.

### Therapeutic Efficacy in Sepsis Mouse Model

Adult CD-1 mice were i.p. administrated with LPS (50 mg/kg). 4 hours later, the mice were grouped randomly (10 mice per group) and i.v. injected with PBS, free DOX and DOX-hyd-BSA NPs (equal to 0.2 mg kg⁻¹ of free DOX), respectively. The healthy mice were used as positive control. At predetermined time points (16 h and 72 h after LPS challenge), mouse BALF, blood and major organs were collected. Cell number and inflammatory factors (TNF-α, IL-6 and IL-1β) in BALF were determined by hemocytometer and ELISA, respectively as aforementioned. Blood was collected as described above. The plasma was harvested for ELISA assay after the blood was centrifuged at 1500 g for 20 min. Cell number was counted as above. The organs were homogenized with PBS (100 mg/mL) to obtain the pipettable homogenate for myeloperoxidase (MPO) activity and ELISA assay.

### Middle Cerebral Artery Occlusion (MCAO) Mouse Model

CD1 mice were used. Mice were anesthetized using 100 mg/kg of ketamine and 5 mg/kg of xylazine. They were positioned in the supine position on a heating pad. A carotid artery (CA) was exposed via the midline neck incision. An external carotid artery (ECA) was separated and occulated with two knots. Next, the internal carotid artery (ICA) was isolated, and the CA and ICA were clipped. A small hole was cut in the ECA above the second knot. A 6-0 medium MCAO suture was then introduced into ICA. Mice were kept for 60 min after occlusion in a heated cage, and the suture was withdrawn for reperfusion. Finally, the skin was closed and the mice were returned to the individual cage.

### Myeloperoxidase (MPO) Activity

The damaged brain tissues were collected 24 h after MCAO and homogenized in PBS with 5% hexadecyltrimethylammonium bromide (HTAB). The homogenate was sonicated, and centrifuged at 13,000 rpm for 10 min. Next, 10 μL supernatant was loaded into each well of a 96-well plate. A solution of o-Dianisidine dihydrochloride with 0.0005% hydrogen peroxide in potassium phosphate buffer was added to the samples. Absorbance was measured at 450 nm. MPO activity is expressed as change in absorbance per minute per gram of tissue.

### Cytokine Quantification

The damaged brain tissues of mice were collected 24 h after MCAO surgery and homogenized in PBS buffer. The level of cytokines (TNF-α, IL-1β, IL-6) was quantified using commercial ELISA assay as aforementioned.

### Assessment of UR Injury by Neurological Deficit Score

Videos were taken 24 h after MCAO surgery and neurological deficit scores were given by two people. The scores are divided into five grades for neurological scores of mice after cerebral ischemia. Grade 0: normal and no neurological defect; grade 1: mild circling when a mouse is picked up via the tail and attempts to rotate to the contralateral side; grade 2: consistent strong and immediate circling, or a mouse only turns to the surgery contralateral side while the animal is suspended by the tail; grade 3: severe rotation or lacking of walking abilities; grade 4: animals do not walk spontaneously and lose the response.

### Statistical Analysis

The experimental data were presented with average values, expressed as the mean±standard deviation (s.d.). Statistical analysis was conducted using one-way ANOVA or Student's t-test of Origin 8.5, p value <0.05 was considered significant.

Accordingly, it has demonstrated herein to selectively target the apoptosis pathway in proinflammatory neutrophils using DOX-conjugated BSA NPs, thus we can manage, for example, sepsis and decrease surgery-induced brain damage in ischemic stroke. The nanoparticle design herein allows controlled release of DOX inside neutrophils, thus avoiding the systemic toxicity. The results discussed demonstrates that DOX can increase neutrophil apoptosis for anti-inflammatory therapies with the beneficial aspect of no cardiac toxicity.

It is to be understood that features described with regard to the various embodiments herein may be mixed and matched in any combination without departing from the spirit and scope of the invention. Although different selected embodiments have been illustrated and described in detail, it is to be appreciated that they are exemplary, and that a variety of substitutions and alterations are possible without departing from the spirit and scope of the present invention.

1. A method of treating a subject with Doxorubicin (DOX) to prevent an activated neutrophil-inflammatory response and transmigration, the method comprising:
administering to the subject, a composition comprising doxorubicin (DOX)-conjugated albumin protein nanoparticles (NPs), wherein the composition includes an effective amount of 0.1 mg/kg up to 10 mg/kg of the doxorubicin (DOX)-conjugated albumin protein nanoparticles (NPs) in vivo, wherein the protein nanoparticles (NPs) selectively bind to and are internalized by the activated neutrophils for intracellular delivery of the doxorubicin (DOX) so as to induce apoptosis of the activated neutrophils; and
wherein a neutrophil induced inflammatory response and transmigration is lower following the administration of the 0.1 mg/kg up to 10 mg/kg of the doxorubicin (DOX)-conjugated albumin protein nanoparticles (NPs).

2. The method of claim 1, wherein the treatment is directed to at least one of: an inflammatory disease, an inflammatory infection, and an inflammatory disorder.

3. The method of claim 1, wherein the treatment is used to prevent an inflammatory response resultant from at least one inflammatory disease, infection, or disorder selected from: a stroke, an aging disorder, cancer, Huntington's disease, encephalitis, autoimmune disorders, immune-complex vasculitis, lupus, cardiomyopathy, ischemic heart disease, atherosclerosis, chronic liver failure, brain and spinal cord trauma, sarcoidosis, arthritis, rheumatoid arthritis, inflammatory bowel disease, ileitis, ulcerative colitis, Barrett's syndrome, Crohn's disease, asthma, onchocerciasis, uveitis, sympathetic ophthalmitis, periodontitis, tuberculosis, glomerulonephritis, nephrosis, sclerodermatitis, psoriasis, eczema, multiple sclerosis, AIDS-related neurodegeneration, Alzheimer's disease, meningitis, encephalomyelitis, Parkinson's disease, acute lung inflammation/injury, and sepsis.

4. The method of claim 1, wherein the composition is administered intravenously.

5. The method of claim 1, wherein the doxorubicin (DOX) is released from the composition in an acidic environment having a pH of 4.0 up to a pH of 6.5.

6. The method of claim 5, wherein the doxorubicin (DOX) and the albumin protein nanoparticles (NPs) are conjugated with hydrazone bonds, and wherein the doxorubicin (DOX) is released by way of a cleavage of the hydrazone bonds in the acidic environment.

7. The method of claim 5, wherein the acidic environment provides for a controlled release of the Doxorubicin (DOX) from the composition, wherein the controlled release avoids systemic toxicity in the subject.

8. The method of claim 1, wherein the administering of the doxorubicin (DOX)-conjugated albumin protein nanoparticles (NPs) decreases cytokine levels.

9. The method of claim 1, wherein the albumin is human serum albumin.

10. The method of claim 1, wherein the albumin is bovine serum albumin.

11. The method of claim 1, wherein the subject is a warm-blooded animal.

12. The method of claim 11, wherein the warm-blooded animal is a human.
